(19) United States
(12) Patent Application Publication
(10) Pub. No.: US 2013/0266567 A1
(43) Pub. Date: Oct. 10, 2013

(54) ANTI-SERUM ALBUMIN BINDING SINGLE VARIABLE DOMAINS

(71) Applicants: Haren Arulanantham, Cambridge (GB); Haiqun Liu, Cambridge (GB); Oliver Schon, Cambridge (GB)

(72) Inventors: Haren Arulanantham, Cambridge (GB); Haiqun Liu, Cambridge (GB); Oliver Schon, Cambridge (GB)

(21) Appl. No.: 13/989,827

(22) PCT Filed: Dec. 1, 2011
(86) PCT No.: PCT/EP11/71497

§ 371 Date: May 28, 2013

Related U.S. Application Data
(60) Provisional application No. 61/418,500, filed on Dec. 1, 2010.

(51) Int. Cl. C07K 16/18

(52) U.S. Cl. CPC C07K 16/18
USPC 424/135.1; 435/320.1; 435/328; 530/387.3; 536/23.53; 530/391.7

(57) ABSTRACT

The invention relates to improved anti-serum albumin immunoglobulin single variable domains, as well as ligands and drug conjugates comprising such variable domains, compositions, nucleic acids, vectors and hosts.

The invention relates to improved anti-serum albumin immunoglobulin single variable domains, as well as ligands and drug conjugates comprising such domains, compositions, nucleic acids, vectors and hosts.

### BACKGROUND OF THE INVENTION

WO04003019 and WO2008/096158 disclose anti-serum albumin (SA) binding moieties, such as anti-SA immunoglobulin single variable domains (dAbs), which have therapeutically-useful half-lives. These documents disclose monomer anti-SA dAbs as well as multi-specific ligands comprising such dAbs, e.g., ligands comprising an anti-SA dAb and a dAb that specifically binds a target antigen, such as TNFR1. Binding moieties are disclosed that specifically bind serum albumins from more than one species, e.g. human/mouse cross-reactive anti-SA dAbs.

WO05118642 and WO2006/059106 disclose the concept of conjugating or associating an anti-SA binding moiety, such as an anti-SA immunoglobulin single variable domain, to a drug, in order to increase the half-life of the drug. Protein, peptide and NCE (chemical entity) drugs are disclosed and exemplified. WO2006/059106 discloses the use of this concept to increase the half-life of insulinotropic agents, e.g., incretin hormones such as glucagon-like peptide (GLP)-1.

Reference is also made to Holt et al, “Anti-Serum albumin domain antibodies for extending the half-lives of short lived drugs”, Protein Engineering, Design & Selection, vol 21, no 5, pp 283-288, 2008.

It would be desirable to provide improved heavy chain variable domain dAbs that specifically bind serum albumin, preferably albumins from human and non-human species, which would provide utility in animal models of disease as well as for human therapy and/or diagnosis. It would also be desirable to provide for the choice between relatively modest- and high-affinity anti-SA binding moieties (dAbs). Such moieties could be linked to drugs, the anti-SA binding moiety being chosen according to the contemplated end-application. This would allow the drug to be better tailored to treating and/or preventing chronic or acute indications, depending upon the choice of anti-SA binding moiety. It would also be desirable to provide anti-SA dAbs that are monomeric or substantially so in solution. This would especially be advantageous when the anti-SA dAb is linked to a binding moiety, e.g., a dAb that specifically binds a cell-surface receptor, such as TNFR1, with the aim of antagonizing the receptor. The monomeric state of the anti-SA dAb is useful in reducing the chance of receptor cross-linking, since multimers are less likely to form which could bind and cross-link receptors (e.g., TNFR1) on the cell surface, thus increasing the likelihood of receptor agonism and detrimental receptor signaling. It would also be desirable to provide anti-SA dAbs that have relatively high melting temperatures. This is useful for providing stable formulations, e.g., storage-stable formulations and variable domains that have a good shelf-life.

### SUMMARY OF THE INVENTION

Aspects of the present invention solve these problems.

In one aspect the invention, therefore, there is provided an anti-serum albumin (SA) immunoglobulin single variable domain comprising an amino acid sequence that is at least 80% identical to an amino acid sequence selected from SEQ ID NOs:1 to 21.

An aspect of the invention provides an anti-serum albumin (SA) immunoglobulin single variable domain comprising an amino acid sequence having up to 4 amino acid changes compared to an amino acid sequence selected from SEQ ID NOs:1 to 21. In one embodiment a variant single variable domain is provided which is identical to said selected domain amino acid sequence with the exception of one, two, three or four amino acid differences. For example, the variable domain comprises or consists of the amino acid sequence of any one of SEQ ID NOs: 1 to 21 (or an amino acid sequence that is at least 95, 96, 97, 98 or 99% identical to the amino acid sequence of any one of SEQ ID NOs: 1 to 21)

An aspect of the invention provides an anti-serum albumin (SA) immunoglobulin single variable domain comprising an amino acid sequence that is encoded by a nucleotide sequence which is at least 80% identical to a sequence selected from SEQ ID NOs25 to 45. In one aspect, the invention provides an anti-serum albumin (SA) immunoglobulin single variable domain selected from DOM7r-31-14, DOM7r-31-100, DOM7r-31-101, DOM7r-31-102, DOM7r-31-103, DOM7r-31-104, DOM7r-36-2, DOM7r-36-100, DOM7r-36-101, DOM7r-36-102, DOM7r-36-103, DOM7r-36-104, DOM7r-36-105, DOM7r-36-106, DOM7r-36-107, DOM7r-36-108, DOM7r-92-4, DOM7r-92-100, DOM7r-92-101, DOM7r-92-102 and DOM7r-92-103.

An aspect of the invention provides a multispecific ligand comprising an anti-SA variable domain of the invention and a binding moiety that specifically binds a target antigen other than SA.

An aspect of the invention provides an anti-SA single variable domain of the invention, wherein the variable domain is conjugated to a drug (optionally an NCE drug).

An aspect of the invention provides a fusion product, e.g., a fusion protein or fusion with a peptide or NCE (new chemical entity) drug, comprising a polypeptide, protein, peptide or NCE drug fused or conjugated (for an NCE) to any anti-SA variable domain of the invention. Suitably, only a modest drop in affinity of the variant for its binding partner is observed when fused or conjugated to a partner making it useful in fusion products.

An aspect of the invention provides a composition comprising a variable domain, fusion protein or ligand of the invention and a pharmaceutically acceptable diluent, carrier, excipient or vehicle.

An aspect of the invention provides a polypeptide fusion or conjugate comprising an anti-serum albumin dAb as disclosed herein and an incretin or insulinotropic agent, e.g., exendin-4, GLP-1 (7-37), GLP-1 (6-36) or any incretin or insulinotropic agent disclosed in WO06/059106, these agents being explicitly incorporated herein by reference as though written herein for inclusion in the present invention and claims below.

In another aspect, the invention provides a multispecific ligand comprising an anti-SA single variable domain of said further aspect and a binding moiety that specifically binds a target antigen other than SA.

An aspect of the invention provides a nucleic acid comprising a nucleotide sequence encoding a variable domain, or a multispecific ligand or fusion protein of the invention.

An aspect of the invention provides a nucleic acid comprising a nucleotide sequence that is at least 80% identical to a sequence selected from SEQ ID NOs 25 to 45.

An aspect of the invention provides a vector comprising the nucleic acid of the invention.

An aspect of the invention provides an isolated host cell comprising the vector of the invention.

An aspect of the invention provides a method of treating or preventing a disease or disorder in a patient, comprising administering at least one dose of a variable domain, or a multispecific ligand or fusion protein of the invention to said patient.

Embodiments of any aspect of the invention provide anti-serum albumin single variable domains of good anti-serum albumin affinities. The choice of variable domain can allow for tailoring of half-life according to the desired therapeutic and/or prophylactic setting. For example, in one embodiment, the affinity of the variable domain for serum albumin is relatively high, such that the variable domain would be useful for inclusion in products that find utility in treating and/or preventing chronic or persistent diseases, conditions, toxicity or other chronic indications. In one embodiment, the affinity of the variable domain for serum albumin is relatively modest, such that the variable domain would be useful for inclusion in products that find utility in treating and/or preventing acute diseases, conditions, toxicity or other acute indications. In one embodiment, the affinity of the variable domain for serum albumin is intermediate, such that the variable domain would be useful for inclusion in products that find utility in treating and/or preventing acute or chronic diseases, conditions, toxicity or other acute or chronic indications.

It is conceivable that a molecule with an appropriately high affinity and specificity for serum albumin would stay in circulation long enough to have the desired therapeutic effect. (Tomlinson, Nature Biotechnology 22, 521-522 (2004)). Here, a high affinity anti-SA variable domain would stay in serum circulation matching that of the species' serum albumin (WO2008096158). Once in circulation, any fused therapeutic agent to the AlbudAb variable domain, be it NCE, peptide or protein, consequently would be able to act longer on its target and exhibit a longer lasting therapeutic effect. This would allow for targeting chronic or persistent diseases without the need of frequent dosing.

A variable domain with moderate affinity, (but specificity to SA) would only stay in serum circulation for a short time (e.g., for a few hours or a few days) allowing for the specific targeting of therapeutic targets involved in acute diseases by the fused therapeutic agent.

This way it is possible to tailor the anti-SA-containing product to the therapeutic disease area by choosing an anti-SA variable domain with the appropriate albumin binding affinity and/or serum half-life.

One of the properties of domain antibodies is that they can exist and bind to target in monomeric or dimeric forms. Other embodiments of any aspect of the invention provide variants which are monomeric or di- or multi-meric. A monomer dAb may be preferred for certain targets or indications where it is advantageous to prevent target cross-linking (for example, where the target is a cell surface receptor such as a receptor tyrosine kinase e.g. TNFR1). In some instances, binding as a dimer or multimer could cause receptor cross-linking of receptors on the cell surface, thus increasing the likelihood of receptor agonism and detrimental receptor signaling. Alternatively, a dAb which forms a dimer may be preferred to ensure target cross-linking or for improved binding through avidity effect, stability or solubility, for example.

For certain targeting approaches involving a multidomain construct, it may be preferable to use a monomer dAb e.g. when a dual targeting molecule is to be generated, such as a dAb-AlbudAb™ where the AlbudAb binds serum albumin, as described above, since dimerizing dAbs may lead to the formation of high molecular weight protein aggregates, for example.

### DETAILED DESCRIPTION OF THE INVENTION

Within this specification the invention has been described, with reference to embodiments, in a way which enables a clear and concise specification to be written. It is intended and should be appreciated that embodiments may be variously combined or separated without parting from the invention.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc. which are incorporated herein by reference) and chemical methods.

A “patient” is any animal, e.g., a mammal, e.g., a non-human primate (such as a baboon, rhesus monkey or Cynomolgus monkey), mouse, human, rabbit, rat, dog, cat or pig. In one embodiment, the patient is a human.

As used herein an antibody refers to IgG, IgM, IgA, IgD or IgE or a fragment (such as a Fab, Fab′, F(ab′)₂, Fv, disulphide linked Fv, scFv, closed conformation multi-specific antibody, disulphide-linked scFv, diabody) whether derived from any species naturally producing an antibody, or created by recombinant DNA technology; whether isolated from serum, B-cells, hybridomas, transfectomas, yeast or bacteria.

As used herein, “antibody format” refers to any suitable polypeptide structure in which one or more antibody variable domains can be incorporated so as to confer binding specificity for antigen on the structure. A variety of suitable antibody formats are known in the art, such as, chimeric antibodies, humanized antibodies, human antibodies, single chain antibodies, bispecific antibodies, antibody heavy chains, antibody light chains, homodimers and heterodimers of antibody heavy chains and/or light chains, antigen-binding fragments of any of the foregoing (e.g., a Fv fragment (e.g., single chain Fv (scFv), a disulfide bonded Fv), a Fab fragment, a Fab′ fragment, a F(ab′)₂ fragment), a single antibody variable domain (e.g., a dAb, V_{H}, V_{HH}, V_{L}), and modified versions of any of the foregoing (e.g., modified by the covalent attachment of polyethylene glycol or other suitable polymer or a humanized V_{HH}).

The phrase “immunoglobulin single variable domain” refers to an antibody variable domain (V_{H}, V_{HH}, V_{L}) that specifically binds an antigen or epitope independently of different V regions or domains. An immunoglobulin single variable domain can be present in a format (e.g., homo- or hetero-multimer) with other variable regions or variable domains where the other regions or domains are not required for antigen binding by the single immunoglobulin variable domain (i.e., where the immunoglobulin single variable domain binds antigen independently of the additional variable domains). A “domain antibody” or “dAb” is the same as an “immunoglobulin single variable domain” as the term is used herein. A “single immunoglobulin variable domain” is the same as an “immunoglobulin single variable domain” as the term is used herein. A “single antibody variable domain” or an “antibody single variable domain” is the same as an “immunoglobulin single variable domain” as the term is used herein. An immunoglobulin single variable domain is in one embodiment a human antibody variable domain, but also includes single antibody variable domains from other species such as rodent (for example, as disclosed in WO 00/29004, the contents of which are incorporated herein by reference in their entirety), nurse shark and CamelidV_{HH} dAbs. Camelid V_{HH} are immunoglobulin single variable domain polypeptides that are derived from species including camel, llama, alpaca, dromedary, and guanaco, which produce heavy chain antibodies naturally devoid of light chains. The V_{HH} may be humanized.

A “domain” is a folded protein structure which has tertiary structure independent of the rest of the protein. Generally, domains are responsible for discrete functional properties of proteins, and in many cases may be added, removed or transferred to other proteins without loss of function of the remainder of the protein and/or of the domain. A “single antibody variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain.

In the instant application, the term “prevention” and “preventing” involves administration of the protective composition prior to the induction of the disease or condition. “Treatment” and “treating” involves administration of the protective composition after disease or condition symptoms become manifest. “Suppression” or “suppressing” refers to administration of the composition after an inductive event, but prior to the clinical appearance of the disease or condition.

As used herein, the term “dose” refers to the quantity of ligand administered to a subject all at one time (unit dose), or in two or more administrations over a defined time interval. For example, dose can refer to the quantity of ligand (e.g., ligand comprising an immunoglobulin single variable domain that binds target antigen) administered to a subject over the course of one day (24 hours) (daily dose), two days, one week, two weeks, three weeks or one or more months (e.g., by a single administration, or by two or more administrations). The interval between doses can be any desired amount of time. The term “pharmaceutically effective” when referring to a dose means sufficient amount of the ligand, domain or pharmaceutically active agent to provide the desired effect. The amount that is “effective” will vary from subject to subject, depending on the age and general condition of the individual, the particular drug or pharmaceutically active agent and the like. Thus, it is not always possible to specify an exact “effective” amount applicable for all patients. However, an appropriate “effective” dose in any individual case may be determined by one of ordinary skill in the art using routine experimentation.

Methods for pharmacokinetic analysis and determination of ligand (e.g., single variable domain, fusion protein or multi-specific ligand) half-life will be familiar to those skilled in the art. Details may be found in Kenneth, A et al: Chemical Stability of Pharmaceuticals: A Handbook for Pharmacists and in Peters et al, Pharmacokinetc analysis: A Practical Approach (1996). Reference is also made to “Pharmacokinetics”, M Gibaldi & D Perron, published by Marcel Dekker, 2^{nd} Rev. ex edition (1982), which describes pharmacokinetic parameters such as t alpha and t beta half lives and area under the curve (AUC). Optionally, all pharmacokinetic parameters and values quoted herein are to be read as being values in a human. Optionally, all pharmacokinetic parameters and values quoted herein are to be read as being values in a mouse or rat or Cynomolgus monkey.

Half lives (t½ alpha and t % beta) and AUC can be determined from a curve of serum concentration of ligand against time. The WinNonlin analysis package, e.g. version 5.1 (available from Pharsight Corp., Mountain View, Calif. 94040, USA) can be used, for example, to model the curve. When two-compartment modeling is used, in a first phase (the alpha phase) the ligand is undergoing mainly distribution in the patient, with some elimination. A second phase (beta phase) is the phase when the ligand has been distributed and the serum concentration is decreasing as the ligand is cleared from the patient. The t alpha half life is the half life of the first phase and the t beta half life is the half life of the second phase. Thus, in one embodiment, in the context of the present invention, the variable domain, fusion protein or ligand has at alpha (or of about) 15 minutes or more. In one embodiment, the lower end of the range is (or is about) 30 minutes, 45 minutes, 1 hour, 2 hours, 3 hours, 4 hours, 5 hours, 6 hours, 7 hours, 10 hours, 11 hours or 12 hours. In addition, or alternatively, the variable domain, fusion protein or ligand according to the invention will have at alpha (or of about) 15 minutes or more. Including 12 hours (or about 12 hours). In one embodiment, the upper end of the range is (or is about) 11, 10, 9, 8, 7, 6 or 5 hours. An example of a suitable range is (or is about) 1 to 6 hours, 2 to 5 hours or 3 to 4 hours.

In one embodiment, the present invention provides the variable domain, fusion protein or ligand according to the invention has at beta (or of about) 2.5 hours or more. In one embodiment, the lower end of the range is (or is about) 3 hours, 4 hours, 5 hours, 6 hours, 7 hours, 10 hours, 11 hours, or 12 hours. In addition, or alternatively, the t beta is (or is about) up to and including 21 or 25 days. In one embodiment, the upper end of the range is (or is about)12 hours, 24 hours, 2 days, 3 days, 5 days, 10 days, 15 days, 19 days 20 days, 21 days or 22 days. For example, the variable domain, fusion protein or ligand according to the invention will have at beta half life in the range 12 to 60 hours (or about 12 to 60 hours). In a further embodiment, it will be in the range 12 to 48 hours (or about 12 to 48 hours). In a further embodiment still, it will be in the range 12 to 26 hours (or about 12 to 26 hours).

As an alternative to using two-compartment modeling, the skilled person will be familiar with the use of non-compartmental modeling, which can be used to determine terminal half-lives (in this respect, the term “terminal half-life” as used herein means a terminal half-life determined using non-compartmental modeling). The WinNonlin analysis package, e.g. version 5.1 (available from Pharsight Corp., Mountain View, Calif. 94040, USA) can be used, for example, to model the curve in this way. In this instance, in one embodiment the single variable domain, fusion protein or ligand has a terminal half life of at least (or at least about) 8 hours, 10 hours, 12 hours, 15 hours, 28 hours, 20 hours, 1 day, 2 days, 3 days, 7 days, 14 days, 15 days, 16 days, 17 days, 18 days, 19 days, 20 days, 21 days, 22 days, 23 days, 24 days or 25 days. In one embodiment, the upper end of this range is (or is about) 24 hours, 48 hours, 60 hours or 72 hours or 120 hours. For example, the terminal half-life is (or is about) from 8 hours to 60 hours, or 8 hours to 48 hours or 12 to 120 hours, e.g., in man.

In addition, or alternatively to the above criteria, the variable domain, fusion protein or ligand according to the invention has an AUC value in addition, or alternatively to the above, of (or of about) 1 mg·min/ml or more. In one embodiment, the lower end of the range is (or is about) 5, 10, 15, 20, 30, 100, 200 or 300 mg·min/ml. In addition, or alternatively, the variable domain, fusion protein or ligand according to the invention has an AUC in the range of (or of about) up to 600 mg·min/ml. In one embodiment, the upper end of the range is (or is about) 500, 400, 300, 200, 150, 100, 75 or 50 mg·min/ml. Advantageously the variable domain, fusion protein or ligand will have an AUC in (or about in) the range selected from the group consisting of the following: 15 to 150 mg·min/ml, 15 to 100 mg·min/ml, 15 to 75 mg·min/ml, and 15 to 50 mg·min/ml.

“Surface Plasmon Resonance”: Competition assays can be used to determine if a specific antigen or epitope, such as human serum albumin, competes with another antigen or epitope, such as cynomolgus serum albumin, for binding to a serum albumin binding ligand described herein, such as a specific dAb. Similarly competition assays can be used to determine if a first ligand such as dAb, competes with a second ligand such as a dAb for binding to a target antigen or epitope. The term “competes” as used herein refers to substance, such as a molecule, compound, preferably a protein, which is able to interfere to any extent with the specific binding interaction between two or more molecules. The phrase “does not competitively inhibit” means that substance, such as a molecule, compound, preferably a protein, does not interfere to any measurable or significant extent with the specific binding interaction between two or more molecules. The specific binding interaction between two or more molecules preferably includes the specific binding interaction between a single variable domain and its cognate partner or target. The interfering or competing molecule can be another single variable domain or it can be a molecule that that is structurally and/or functionally similar to a cognate partner or target.

The term “binding moiety” refers to a domain that specifically binds an antigen or epitope independently of a different epitope or antigen binding domain. A binding moiety may be a domain antibody (dAb) or may be a domain which is a derivative of a non-immunoglobulin protein scaffold, e.g., a scaffold selected from the group consisting of CTLA-4, lipocalin, SpA, an adnectin, affibody, an avimer, GroEI, transferrin, GroES and fibronectin, which binds to a ligand other than the natural ligand (in the case of the present invention, the moiety binds serum albumin). See WO2008/096158, which discloses examples of protein scaffolds and methods for selecting antigen or epitope-specific binding domains from repertoires (see Examples 17 to 25). These specific disclosures of WO2008/096158 are expressly incorporated herein by reference as though explicitly written herein and for use with the present invention, and it is contemplated that any part of such disclosure can be incorporated into one or more claims herein).

In one embodiment, a variable domain of the invention comprises one or more of the following kinetic characteristics:—
(a) The variable domain comprises a binding site that specifically binds human SA with a dissociation constant (KD) from (or from about) 0.1 to (or to about) 10000 nM, optionally from (or from about) 1 to (or to about) 6000 nM, as determined by surface plasmon resonance;
(b) The variable domain comprises a binding site that specifically binds human SA with an off-rate constant (K_{d}) from (or from about) 1.5×10⁻⁴ to (or to about) 0.1 sec⁻¹, optionally from (or from about) 3×10⁻⁴ to (or to about) 0.1 seq⁻¹ as determined by surface plasmon resonance;
(c) The variable domain comprises a binding site that specifically binds human SA with an on-rate constant (Kₐ) from (or from about)2×10⁶ to (or to about) 1×10⁴M⁻¹sec⁻¹, optionally from (or from about) 1×10⁶ to (or to about) 2×10⁴ M⁻¹ sec⁻¹ as determined by surface plasmon resonance;
(d) The variable domain comprises a binding site that specifically binds Cynomolgus monkey SA with a dissociation constant (KD) from (or from about) 0.1 to (or to about) 10000 nM, optionally from (or from about) 1 to (or to about) 6000 nM, as determined by surface plasmon resonance;
(e) The variable domain of any preceding claim, wherein the variable domain comprises a binding site that specifically binds Cynomolgus monkey SA with an off-rate constant (K_{d}) from (or from about) 1.5×10⁻⁴ to (or to about) 0.1 sec⁻¹, optionally from (or from about) 3×10⁻⁴ to (or to about) 0.1 seq⁻¹ as determined by surface plasmon resonance;
(f) The variable domain of any preceding claim, wherein the variable domain comprises a binding site that specifically binds Cynomolgus monkey SA with an on-rate constant (Kₐ) from (or from about) 2×10⁶ to (or to about) 1×10⁴M⁻¹sec⁻¹, optionally from (or from about) 1×10⁶ to (or to about) 5×10³ M⁻¹ sec⁻¹ as determined by surface plasmon resonance;
(g) The variable domain comprises a binding site that specifically binds rat SA with a dissociation constant (KD) from (or from about) 1 to (or to about) 10000 nM, optionally from (or from about) 20 to (or to about) 6000 nM, as determined by surface plasmon resonance;
(h) The variable domain comprises a binding site that specifically binds rat SA with an off-rate constant (K_{d}) from (or from about) 2×10⁻³ to (or to about) 0.15 sec⁻¹, optionally from (or from about) 9×10⁻³ to (or to about) 0.14 sec⁻¹ as determined by surface plasmon resonance;
(i) The variable domain comprises a binding site that specifically binds rat SA with an on-rate constant (kₐ) from (or from about) 2×10⁶ to (or to about) 1×10⁴M⁻¹sec⁻¹, optionally from (or from about) 1×10⁶ to (or to about) 3×10⁴M⁻¹sec⁻¹ as determined by surface plasmon resonance;
(j) The variable domain comprises a binding site that specifically binds mouse SA with an equilibrium dissociation constant (KD) from (or from about) 1 to (or to about) 10000 nM as determined by surface plasmon resonance;
(k) The variable domain comprises a binding site that specifically binds mouse SA with an off-rate constant (k_{d}) from (or from about) 2×10⁻³ to (or to about) 0.15 sec⁻¹ as determined by surface plasmon resonance; and/or
(l) The variable domain comprises a binding site that specifically binds mouse SA with an on-rate constant (kₐ) from (or from about) 2×10⁶ to (or to about) 1×10⁴M⁻¹sec⁻¹, optionally from (or from about) 2×10⁶ to (or to about) 1.5×10⁴M⁻¹sec⁻¹ as determined by surface plasmon resonance.
Optionally, the variable domain has
I: a KD according to (a) and (d), a k_{d} according to (b) and (e), and a kₐ according to (c) and (f); or
II: a KD according to (a) and (g), a k_{d} according to (b) and (h), and a kₐ according to (c) and (i); or
III: a KD according to (a) and (j), a k_{d} according to (b) and (k), and a kₐ according to (c) and (I); or
IV: kinetics according to I and II; or
V: kinetics according to I and III; or
VI: kinetics according to I, II and III.

The invention also provides a ligand comprising a variable domain of any preceding aspect or embodiment of the invention. For example, the ligand can be a dual-specific ligand (see WO04003019 for examples of dual-specific ligands). In one aspect, the invention provides a multispecific ligand comprising an anti-SA variable domain of any preceding aspect or embodiment of the invention and a binding moiety that specifically binds a target antigen other than SA. The binding moiety can be any binding moiety that specifically binds a target, e.g., the moiety is an antibody, such as a MAb, an antibody fragment, scFv, Fab, dAb or a binding moiety comprising a non-immunoglobulin protein scaffold. Such moieties are disclosed in detail in WO2008/096158 (see examples 17 to 25, which disclosure is incorporated herein by reference). Examples of non-immunoglobulin scaffolds are CTLA-4, lipocallin, staphylococcal protein A (spA), Affibody™, Avimers™ adnectins, GroEL and fibronectin.

In one embodiment, a linker is provided between the anti-target binding moiety and the anti-SA variable domain, the linker comprising the amino acid sequence AST, optionally ASTSGPS. Alternative linkers are described in Huston et al., 1988, PNAS 85:5879-5883; Wright & Deonarain, Mol. Immunol, 2007, 44:2860-2869; Alfthan et al, Prot. Eng., 1995, 8:725-731; Luo et ai, J. Biochem., 1995, 118:825-831; Tang et al, 1996, J. Biol. Chem. 271:15682-15686; Turner et al, 1997, JIMM 205, 42-54 (see Table 1 for representative examples); WO 2009040562; WO2007085814 and WO2008096158 (see the passage at page 135, line 12 to page 140, line 14), all of which references and all disclosed sequences of linkers are expressly incorporated herein by reference as though explicitly written herein and for use with the present invention, and it is contemplated that any part of such disclosure can be incorporated into one or more claims herein.

As used herein, the term “antagonist of Tumor Necrosis Factor Receptor 1 (TNFR1)” or “anti-TNFR1 antagonist” or the like refers to an agent (e.g., a molecule, a compound) which binds TNFR1 and can inhibit a (i.e., one or more) function of TNFR1. For example, an antagonist of TNFR1 can inhibit the binding of TNF alpha to TNFR1 and/or inhibit signal transduction mediated through TNFR1. Accordingly, TNFR1-mediated processes and cellular responses (e.g., TNF alpha—induced cell death in a standard L929 cytotoxicity assay) can be inhibited with an antagonist of TNFR1.

In one embodiment, the multispecific ligand comprises an anti-SA dAb variant or moiety of the invention and an anti-TNFR1 binding moiety, e.g., an anti-TNFR1 dAb. Optionally, the ligand has only one anti-TNFR1 binding moiety (e.g., dAb) to reduce the chance of receptor cross-linking. Anti-TNFR1 dAbs are described, for example, in WO2006/038027, WO2007/049017, WO2008149148 and WO2010/081787 (the amino acid sequences of which and the nucleotide sequence of which, as disclosed in those PCT applications, are expressly incorporated herein by reference as though explicitly written herein and for use with the present invention, and it is contemplated that any part of such disclosures can be incorporated into one or more claims herein).

In one embodiment, the multispecific ligand comprises an anti-SA dAb variable domain of the invention and an anti-TNFR1 binding moiety, e.g., an anti-TNFR1 dAb. Optionally, the ligand has only one anti-TNFR1 binding moiety (e.g., dAb) to reduce the chance of receptor cross-linking.

In one embodiment, the ligand of the invention is a fusion protein comprising a variant or moiety of the invention fused directly or indirectly to one or more polypeptides. For example, the fusion protein can be a “drug fusion” as disclosed in WO2005/118642 (the disclosure of which is incorporated herein by reference), comprising a variant or moiety of the invention and a polypeptide drug as defined in that PCT application.

In one embodiment of the multispecific ligand, the target antigen may be, or be part of, polypeptides, proteins or nucleic acids, which may be naturally occurring or synthetic. In this respect, the ligand of the invention may bind the target antigen and act as an antagonist or agonist (e.g., EPO receptor agonist). One skilled in the art will appreciate that the choice is large and varied. They may be for instance, human or animal proteins, cytokines, cytokine receptors, where cytokine receptors include receptors for cytokines, enzymes, co-factors for enzymes or DNA binding proteins. Suitable cytokines and growth factors include, but are preferably not limited to: ApoE, Apo-SAA, BDNF, Cardiotrophin-1, EGF, EGF receptor, ENA-78, Eotaxin, Eotaxin-2, Exodus-2, EpoR, FGF-acidic, FGF-basic, fibroblast growth factor-10, FLT3 ligand, Fractalkine (CX3C), GDNF, G-CSF, GM-CSF, GF-β1, insulin, IFN-γ, IGF-I, IGF-II, IL-1α, IL-1β, IL-2, IL-3, IL-4, IL-5, IL-6, IL-7, IL-8 (72 a.a.), IL-8 (77 a.a.), IL-9, IL-10, IL-11, IL-12, IL-β, IL-15, IL-16, IL-17, IL-18 (IGIF), Inhibin α, Inhibin β, IP-10, keratinocyte growth factor-2 (KGF-2), KGF, Leptin, LIF, Lymphotactin, Mullerian inhibitory substance, monocyte colony inhibitory factor, monocyte attractant protein, M-CSF, MDC (67 a.a.), MDC (69 a.a.), MCP-1 (MCAF), MCP-2, MCP-3, MCP-4, MDC (67 a.a.), MDC (69 a.a.), MIG, MIP-1α, MIP-3α, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TGF-α, TGF-β2, TGF-β3, tumour necrosis factor (TNF), TNF-α, TNF-β, TNF receptor I, TNF receptor II, TNIL-1, TPO, VEGF, VEGF receptor 1, VEGF receptor 2, VEGF receptor 3, GCP-2, GRO/MGSA, GRO-β, GRO-γ, HCC1, 1-309, HER 1, HER 2, HER 3 and HER 4, CD4, human chemokine receptors CXCR4 or CCR5, non-structural protein type 3 (NS3) from the hepatitis C virus, TNF-alpha, IgE, IFN-gamma, MMP-12, CEA, H. pylori, TB, influenza, Hepatitis E, MMP-12, internalizing receptors that are over-expressed on certain cells, such as the epidermal growth factor receptor (EGFR), ErBb2 receptor on tumor cells, an internalising cellular receptor, LDL receptor, FGF2 receptor, ErbB2 receptor, transferrin receptor, PDGF receptor, VEGF receptor, PsmAr, an extracellular matrix protein, elastin, fibronectin, laminin, □HCC1, 1-309, HER 1, HER 2, HER 3 and HER 4, CD4, human chemokine receptors CXCR4 or CCR5, non-structural protein type 3 (NS3) from the hepatitis C virus, TNF-alpha, IgE, IFN-gamma, MMP-12, virus. It will be appreciated that this list is by no means exhaustive.

As used herein, “drug” refers to any compound (e.g., small organic molecule, nucleic acid, polypeptide) that can be administered to an individual to produce a beneficial, therapeutic or diagnostic effect through binding to and/or altering the function of a biological target molecule in the individual. The target molecule can be an endogenous target molecule encoded by the individual's genome (e.g. an enzyme, receptor, growth factor, cytokine encoded by the individual's genome) or an exogenous target molecule encoded by the genome of a pathogen (e.g. an enzyme encoded by the genome of a virus, bacterium, fungus, nematode or other pathogen). Suitable drugs for use in fusion proteins and conjugates comprising an anti-SA dAb domain of the invention are disclosed in WO2005/118642 and WO2006/059106 (the entire disclosures of which are incorporated herein by reference, and including the entire list of specific drugs as though this list were expressly written herein, and it is contemplated that such incorporation provides disclosure of specific drugs for inclusion in claims herein). For example, the drug can be glucagon-like peptide 1 (GLP-1) or a variant, interferon alpha 2b or a variant or exendin-4 or a variant.

In one embodiment, the invention provides a drug conjugate as defined and disclosed in WO2005/118642 and WO2006/059106, wherein the conjugate comprises a variable domain of the invention. In one example, the drug is covalently linked to the variable domain (e.g., the variable domain and the drug are expressed as part of a single polypeptide). Alternatively, in an example, the drug is non-covalently bonded or associated with the variable domain. The drug can be covalently or noncovalently bonded to the variable domain directly or indirectly (e.g., through a suitable linker and/or noncovalent binding of complementary binding partners (e.g., biotin and avidin)). When complementary binding partners are employed, one of the binding partners can be covalently bonded to the drug directly or through a suitable linker moiety, and the complementary binding partner can be covalently bonded to the variable domain directly or through a suitable linker moiety. When the drug is a polypeptide or peptide, the drug composition can be a fusion protein, wherein the polypeptide or peptide, drug and the polypeptide binding moiety are discrete parts (moieties) of a continuous polypeptide chain. As described herein, the polypeptide binding moieties and polypeptide drug moieties can be directly bonded to each other through a peptide bond, or linked through a suitable amino acid, or peptide or polypeptide linker.

A ligand which contains one single variable domain (e.g., monomer) of the invention or more than one single variable domain (multimer, fusion protein, conjugate, and dual specific ligand as defined herein) which specifically binds to serum albumin, can further comprise one or more entities selected from, but preferably not limited to a label, a tag, an additional single variable domain, a dAb, an antibody, and antibody fragment, a marker and a drug. One or more of these entities can be located at either the COOH terminus or at the N terminus or at both the N terminus and the COOH terminus of the ligand comprising the single variable domain, (either immunoglobulin or non-immunoglobulin single variable domain). One or more of these entities can be located at either the COOH terminus, or the N terminus, or both the N terminus and the COOH terminus of the single variable domain which specifically binds serum albumin of the ligand which contains one single variable domain (monomer) or more than one single variable domains (multimer, fusion protein, conjugate, and dual specific ligand as defined herein). Non-limiting examples of tags which can be positioned at one or both of these termini include a HA, his or a myc tag. The entities, including one or more tags, labels and drugs, can be bound to the ligand which contains one single variable domain (monomer) or more than one single variable domain (multimer, fusion protein, conjugate, and dual specific ligand as defined herein), which binds serum albumin, either directly or through linkers as described above.

An aspect of the invention provides a fusion product, e.g., a fusion protein or fusion with a peptide or conjugate with an NCE (new chemical entity) drug, comprising a polypeptide drug fused or conjugated (for an NCE) to any variable domain as described above.

The invention provides a composition comprising a variable domain, fusion protein, conjugate or ligand of any aspect of the invention and a pharmaceutically acceptable diluent, carrier, excipient or vehicle.

Also encompassed herein is an isolated nucleic acid encoding any of the variable domain, fusion proteins, conjugates or ligands described herein, e.g., a ligand which contains one single variable domain (e.g., monomer) of the invention or more than one single variable domain (e.g., multimer, fusion protein, conjugate, and dual specific ligand as defined herein) which specifically binds to serum albumin, or which specifically binds both human serum albumin and at least one non-human serum albumin, or functionally active fragments thereof. Also encompassed herein is a vector and/or an expression vector, a host cell (e.g., a non-human host cell or a host cell that is not isolated from a human or human embryo) comprising the vector, e.g., a plant or animal cell and/or cell line transformed with a vector, a method of expressing and/or producing one or more variable domains, fusion proteins or ligands which contains one single variable domain (monomer) or more than one single variable domains (e.g., multimer, fusion protein, conjugate, and dual specific ligand as defined herein) which specifically binds to serum albumin, or fragment(s) thereof encoded by said vectors, including in some instances culturing the host cell so that the one or more variable domains, fusion proteins or ligands or fragments thereof are expressed and optionally recovering the ligand which contains one single variable domain (monomer) or more than one single variable domain (e.g., multimer, fusion protein, conjugate, and dual specific ligand as defined herein) which specifically binds to serum albumin, from the host cell culture medium. Also encompassed are methods of contacting a ligand described herein with serum albumin, including serum albumin and/or non-human serum albumin(s), and/or one or more targets other than serum albumin, where the targets include biologically active molecules, and include animal proteins, cytokines as listed above, and include methods where the contacting is in vitro as well as administering any of the variable domains, fusion proteins or ligands described herein to an individual host animal or cell in vivo and/or ex vivo. Preferably, administering ligands described herein which comprises a single variable domain (immunoglobulin or non-immunoglobulin) directed to serum albumin and/or non-human serum albumin(s), and one or more domains directed to one or more targets other than serum albumin, will increase the half life, including the T beta and/or terminal half life, of the anti-target ligand. Nucleic acid molecules encoding the domains, fusion proteins or single domain containing ligands or fragments thereof, including functional fragments thereof, are contemplated herein. Vectors encoding the nucleic acid molecules, including but preferably not limited to expression vectors, are contemplated herein, as are host cells from a cell line or organism containing one or more of these expression vectors. Also contemplated are methods of producing any domain, fusion protein or ligand, including, but preferably not limited to any of the aforementioned nucleic acids, vectors and host cells.

An aspect of the invention provides a nucleic acid comprising a nucleotide sequence encoding a variable domain according to the invention or a multispecific ligand of the invention or fusion protein of the invention.

An aspect of the invention provides a nucleic acid comprising the nucleotide sequence selected from of any one of SEQ ID NOs: 25 to 45, or a nucleotide sequence that is at least 70, 75, 80, 85, 90, 95, 96, 97, 98 or 99% identical to said selected sequence.

An aspect of the invention provides a vector comprising the nucleic acid of the invention. An aspect of the invention provides an isolated host cell comprising the vector.

Reference is made to WO2008/096158 for details of library vector systems, combining single variable domains, characterization of dual specific ligands, structure of dual specific ligands, scaffolds for use in constructing dual specific ligands, uses of anti-serum albumin dAbs and multispecific ligands and half-life-enhanced ligands, and compositions and formulations of comprising anti-serum albumin dAbs. These disclosures are incorporated herein by reference to provide guidance for use with the present invention, including for domains, ligands, fusion proteins, conjugates, nucleic acids, vectors, hosts and compositions of the present invention.

### Sequences of Anti-Serum Albumin VH Single Variable Domains

Amino acid sequences:
DOM7r-31-14
(SEQ ID NO: 1)
EVQLLESGGGLVQPGGSLRLSCTASGFTFRHYRMGWVRQAPGKGLEWVSWIRPDGTFT
YYADSVKGRFTISRDNSKNTLYLQMNSLRAEDTAVYYCAKSYMADRFDYWGQGTLVTVS
S
DOM7r-31-100
(SEQ ID NO: 2)
EVQLLEPGGGLVQPGGSLRLSCTASGFTFRHYRMGWVRQAPGKGLEWVSWIRPDGTFT
YYADSVKGRFTISRDNSKNTLYLQMNSLRAEDTAVYYCAKSYLSGTFDYWGQGTLVTVSS
DOM7r-31-101
(SEQ ID NO: 3)
EVQLLESGGGLVQPGGSLRLSCTASGFTFRHYRMGWVRQAPGKGLEWVSWIRPDGTFT
YYADSVKGRFTISRDNSKNTLYLQMNSLRAEDTAVYYCAKSYQAGTFDYWGQGTLVTVS
S
DOM7r-31-102
(SEQ ID NO: 4)
EVQLLESGGGLVQPGGSLRLSCTASGFTFRHYRMGWVRQAPGKGLEWVSWIRPDGTFT
YYADSVKGRFTISRDNSKNTLYLQMNSLRAEDTAVYYCAKSYHADKFDYWGQGTLVTVS
S
DOM7r-31-103
(SEQ ID NO: 5)
EVQLLESGGGLVQPGGSLRLSCTASGFTFRHYRMGWVRQAPGKGLEWVSWIRPDGTFT
YYADSVKGRFTISRDNSKNTLYLQMNSLRAEDAAVYYCAKSYMSGTFDYWGQGTLVTVS
S
DOM7r-31-104
(SEQ ID NO: 6)
EVQLLESGGGLVQPGGSLRLSCTASGFTFRHYRMGWVRQAPGKGLEWVSWIRPDGTFT
YYADSVKGRFTISRDNSKNTLYLQMNSLRAEDTAVYYCAKSYMAGTFDYWGQGTLVTVS
S
DOM7r-36-2
(SEQ ID NO: 7)
EVQLLESGGGLVQPGGSLRLSCAASGFTFNHYTMGWVRQAPGKGREWVSLIHPSGTVT
YYADSVKGRFTISRDNSKNTLYLQMNSLRAEDTAVYYCAKWSSRAFDYWGQGTLVTVSS
DOM7r-36-100
(SEQ ID NO: 8)
EVQLLESGGGLVQPGGSLRLSCAASGFTFNHYTMGWVRQAPGKGREWVSLIHPSGTVT
YYADSVKGRFTISRDNSKNTLYLQMNSLRAEDTAVYYCAKWERRTFDYWGQGTLVTVSS
DOM7r-36-101
(SEQ ID NO: 9)
EVQLLESGGGLVQPGGSLRLSCAASGFTFNHYTMGWVRQAPGKGREWVSLIHPSGTVT
YYADSVKGRFTISRDNSKNTLYLQMNSLRAEDTAVYYCAKWFLKSFDYWGQGTLVTVSS
DOM7r-36-102
(SEQ ID NO: 10)
EVQLLESGGGLVQPGGSLRLSCAASGFTFNHYTMGWVRQAPGKGREWVSLIHPSGTVT
YYADSVKGRFTISRDNSKNTLYLQMNSLRAEDTAVYYCAKWFYRNFDYWGQGTLVTVSS
DOM7r-36-103
(SEQ ID NO: 11)
EVQLLESGGGLVQPGGSLRLSCAASGFTFNHYTMGWVRQAPGKGREWVSLIHPSGTVT
YYADSVKGRFTTSRDNSKNTLYLQMNSLRAEDTAVYYCAKWDRKLFDHWGQGTLVTVSS
DOM7r-36-104
(SEQ ID NO: 12)
EVQLLESGGGLVQPGGSLRLSCAASGFTFNHYTMGWVRQAPGKGREWVSLIHPSGTVT
YYADSVKGRFTISRDNSKNTLYLQMNSLRAEDTAVYYCAKWATRGFDYWGQGTLVTVSS
DOM7r-36-105
(SEQ ID NO: 13)
EVQLLESGGGLVQPGGSLRLSCAASGFTFNHYTMGWVRQAPGKGREWVSLIHPSGTVT
YYADSVKGRFTISRDNSKNTLYLQMNSLRAEDTAVYYCAKLGTKVFDYWGQGTLVTVSS
DOM7r-36-106
(SEQ ID NO: 14)
EVQLLESGGGLVQPGGSLRLSCAASGFTFNHYTMGWVRQAPGKGREWVSLIHPSGTVT
YYADSVKGRFTISRDNSKNTLYLQMNSLRAEDTAVYYCAKFRGRGFDYWGQGTLVTVSS
DOM7r-36-107
(SEQ ID NO: 15)
EVQLLESGGGLVQPGGSLRLSCAASGFTFNHYTMGWVRQAPGKGREWVSLIHPSGTVT
YYADSVKGRFTISRDNSKNTLYLQMNSLRAEDTAVYYCAKWEKKYFDYWGQGTLVTVSS
DOM7r-36-108
(SEQ ID NO: 16)
EVQLLESGGGLVQPGGSLRLSCAASGFTFNHYTMGWVRQAPGKGREWVSLIHPSGTVT
YYADSVKGRFTTSRDNSKNTLYLQMNSLRAEDTAVYYCAKLEGRSFDYWGQGTLVTVSS
DOM7r-92-4
(SEQ ID NO: 17)
EVQLLESGGGLVQPGGSLRLSCAASGFTFDTSSMLWVRQAPGKGLEWVSVIHQSGTPTY
YADSVKGRFTISRDNSKNTLYLQMNSLRAEDTAVYYCAKFPSTHGKFDYWGQGTLVTVSS
DOM7r-92-100
(SEQ ID NO: 18)
EVQLLESGGGLVQPGGSLRLSCAASGFTFDTSSMLWVRQAPGKGLEWVSVIHQSGTPTY
YADSVKGRFTISRDNSKNTLYLQMNSLRAEDTAVYYCAKFPSSRMKFDYWGQGTLVTVS
S
DOM7r-92-101
(SEQ ID NO: 19)
EVQLLESGGGLVQPGGSLRLSCAASGFTFDTSSMLWVRQAPGKGLEWVSVIHQSGTPTY
YADSVKGRFTISRDNSKNTLYLQMNSLRAEDTAVYYCAKFPSRKMKFDYRGQGTLVTVSS
DOM7r-92-102
(SEQ ID NO: 20)
EVQLLESGGGLVQPGGSLRLSCAASGFTFDTSSMLWVRQAPGKGLEWVSVIHQSGTPTY
YADSVKGRFTISRDNSKNTLYLQMNSLRAEDTAVYYCAKFPSSQFRFDYWGQGTLVTVSS
DOM7r-92-103
(SEQ ID NO: 21)
EVQLLESGGGLVQPGGSLRLSCAASGFTFDTSSMLWVRQAPGKGLEWVSVIHQSGTPTY
YADSVKGRFTISRDNSKNTLYLQMNSLRAEDTAVYYCAKFPSEGMNFDYWGQGTLVTVS
S
DOM7r-31
(SEQ ID NO: 22)
EVQLLESGGGLVQPGGSLRLSCTASGFTFRHYRMGWVRQAPGKGLEWVSWIRPDGTFT
YYADSVKGRFTISRDNSKNTLYLQMNSLRAEDTAVYYCAKSYMGDRFDYWGQGTLVTVS
S
DOM7r-36
(SEQ ID NO: 23)
EVQLLESGGGLVQPGGSLRLSCAASGFTFNHYTMGWVRQAPGKGLEWVSLIHPSGTVTY
YADSVKGRFTISRDNSKNTLYLQMNSLRAEDTAVYYCAKWSSRAFDYWGQGTLVTVSS
DOM7r-92
(SEQ ID NO: 24)
EVQLLESGGGLVQPGGSLRLSCAASGFTFDTSSMLWVRQAPGKGLEWVSVIHQSGTPTY
YADSVKGRFTISRDNSKNTLYLQMNSLRAEDTAVYYCAKFPFTHGKFDYWGQGTLVTVSS
Nucleotide sequences:
DOM7r-31-14
(SEQ ID NO: 25)
GAGGTGCAGCTGTTGGAGTCTGGGGGAGGCTTGGTACAGCCTGGGGGGTCCCTGCG
TCTCTCCTGTACAGCCTCCGGATTCACCTTTAGGCATTATCGTATGGGTTGGGTCCGC
CAGGCTCCAGGGAAGGGTCTAGAGTGGGTCTCATGGATTCGTCCGGATGGTACGTTT
ACATACTACGCAGACTCCGTGAAGGGCCGGTTCACCATCTCCCGCGACAATTCCAAG
AACACGCTGTATCTGCAAATGAACAGCCTGCGTGCCGAGGACACCGCGGTATATTACT
GTGCGAAATCTTATATGGCTGATAGGTTTGACTACTGGGGTCAGGGAACCCTGGTCAC
CGTCTCGAGC
DOM7r-31-100
(SEQ ID NO: 26)
GAGGTGCAGCTGTTGGAGCCTGGGGGAGGCTTGGTACAGCCTGGGGGGTCCCTGCG
TCTCTCCTGTACAGCCTCCGGATTCACCTTTAGGCATTATCGTATGGGTTGGGTCCGC
CAGGCTCCAGGGAAGGGTCTAGAGTGGGTCTCATGGATTCGTCCGGATGGTACGTTT
ACATACTACGCAGACTCCGTGAAGGGCCGGTTCACCATCTCCCGCGACAATTCCAAG
AACACGCTGTATCTGCAAATGAACAGCCTGCGTGCCGAGGACACCGCGGTATATTACT
GTGCGAAATCTTATCTTAGTGGTACTTTTGACTACTGGGGTCAGGGAACCCTGGTCAC
CGTCTCGAGC
DOM7r-31-101
(SEQ ID NO: 27)
GAGGTGCAGCTGTTGGAGTCTGGGGGAGGCCTGGTACAGCCTGGGGGGTCCCTGCG
TCTCTCCTGTACAGCCTCCGGATTCACCTTTAGGCATTATCGTATGGGTTGGGTCCGC
CAGGCTCCAGGGAAGGGTCTAGAGTGGGTCTCATGGATTCGTCCGGATGGTACGTTT
ACATACTACGCAGACTCCGTGAAGGGCCGGTTCACCATCTCCCGCGACAATTCCAAG
AACACGCTGTATCTGCAAATGAACAGCCTGCGTGCCGAGGACACCGCGGTATATTACT
GTGCGAAATCTTATCAGGCTGGTACGTTTGACTACTGGGGTCAGGGAACCCTGGTCA
CCGTCTCGAGC
DOM7r-31-102
(SEQ ID NO: 28)
GAGGTGCAGCTGTTGGAGTCTGGGGGAGGCTTGGTACAGCCTGGGGGGTCCCTGCG
TCTCTCCTGTACAGCCTCCGGATTCACCTTTAGGCATTATCGTATGGGTTGGGTCCGC
CAGGCTCCAGGGAAGGGTCTAGAGTGGGTCTCATGGATTCGTCCGGATGGTACGTTT
ACATACTACGCAGACTCCGTGAAGGGCCGGTTCACCATCTCCCGCGACAATTCCAAG
AACACGCTGTATCTGCAAATGAACAGCCTGCGTGCCGAGGACACCGCGGTATATTACT
GTGCGAAATCTTATCATGCGGATAAGTTTGACTACTGGGGTCAGGGAACCCTGGTCAC
CGTCTCGAGC
DOM7r-31-103
(SEQ ID NO: 29)
GAGGTGCAGCTGTTGGAGTCTGGGGGAGGCTTGGTACAGCCTGGGGGGTCCCTGCG
TCTCTCCTGTACAGCCTCCGGATTCACCTTTAGGCATTATCGTATGGGTTGGGTCCGC
CAGGCTCCAGGGAAGGGTCTAGAGTGGGTCTCATGGATTCGTCCGGATGGTACGTTT
ACATACTACGCAGACTCCGTGAAGGGCCGGTTCACCATCTCCCGCGACAATTCCAAG
AACACGCTGTATCTGCAAATGAACAGCCTGCGTGCCGAGGACGCTGCGGTATATTACT
GTGCGAAATCTTATATGTCTGGTACTTTTGACTACTGGGGTCAGGGAACCCTGGTCAC
CGTCTCGAGC
DOM7r-31-104
(SEQ ID NO: 30)
GAGGTGCAGCTGTTGGAGTCTGGGGGAGGCTTGGTACAGCCTGGGGGGTCCCTGCG
TCTCTCCTGTACAGCCTCCGGATTCACCTTTAGGCATTATCGTATGGGTTGGGTCCGC
CAGGCTCCAGGGAAGGGTCTAGAGTGGGTCTCATGGATTCGTCCGGATGGTACGTTT
ACATACTACGCAGACTCCGTGAAGGGCCGGTTCACCATCTCCCGCGACAATTCCAAG
AACACGCTGTATCTGCAAATGAACAGCCTGCGTGCCGAGGACACTGCGGTATATTACT
GTGCGAAATCTTATATGGCTGGGACGTTTGACTACTGGGGTCAGGGAACCCTGGTCA
CCGTCTCGAGC
DOM7r-36-2
(SEQ ID NO: 31)
GAGGTGCAGCTGTTGGAGTCTGGGGGAGGCTTGGTACAGCCTGGGGGGTCCCTGCG
TCTCTCCTGTGCAGCCTCCGGATTCACCTTTAATCATTATACGATGGGGTGGGTCCGC
CAGGCTCCAGGGAAGGGTCGAGAGTGGGTCTCATTGATTCATCCGAGTGGTACGGTG
ACATACTACGCAGACTCCGTGAAGGGCCGGTTCACCATCTCCCGCGACAATTCCAAG
AACACGCTGTATCTGCAAATGAACAGCCTGCGTGCCGAGGACACCGCGGTATATTACT
GTGCGAAATGGAGTTCGAGGGCGTTTGACTACTGGGGTCAGGGAACCCTGGTCACCG
TCTCGAGC
DOM7r-36-100
(SEQ ID NO: 32)
GAGGTGCAGCTGTTGGAGTCTGGGGGAGGCTTGGTACAGCCTGGGGGGTCCCTGCG
TCTCTCCTGTGCAGCCTCCGGATTCACCTTTAATCATTATACGATGGGGTGGGTCCGC
CAGGCTCCAGGGAAGGGTCGAGAGTGGGTCTCATTGATTCATCCGAGTGGTACGGTG
ACATACTACGCAGACTCCGTGAAGGGCCGGTTCACCATCTCCCGCGACAATTCCAAG
AACACGCTGTATCTGCAAATGAACAGCCTGCGTGCCGAGGACACCGCGGTATATTACT
GTGCGAAATGGGAGAGGCGGACTTTTGACTACTGGGGTCAGGGAACCCTGGTCACC
GTCTCGAGC
DOM7r-36-101
(SEQ ID NO: 33)
GAGGTGCAGCTGTTGGAGTCTGGGGGAGGCTTGGTACAGCCTGGGGGGTCCCTGCG
TCTCTCCTGTGCAGCCTCCGGATTCACCTTTAATCATTATACGATGGGGTGGGTCCGC
CAGGCTCCAGGGAAGGGTCGAGAGTGGGTCTCATTGATTCATCCGAGTGGTACGGTG
ACATACTACGCAGACTCCGTGAAGGGCCGGTTCACCATCTCCCGCGACAATTCCAAG
AACACGCTGTATCTGCAAATGAACAGCCTGCGTGCCGAGGACACCGCGGTATATTACT
GTGCGAAATGGTTTCTGAAGAGTTTTGACTACTGGGGTCAGGGAACCCTGGTCACCG
TCTCGAGC
DOM7r-36-102
(SEQ ID NO: 34)
GAGGTGCAGCTGTTGGAGTCTGGGGGAGGCTTGGTACAGCCTGGGGGGTCCCTGCG
TCTCTCCTGTGCAGCCTCCGGATTCACCTTTAATCATTATACGATGGGGTGGGTCCGC
CAGGCTCCAGGGAAGGGTCGAGAGTGGGTCTCATTGATTCACCCGAGTGGTACGGTG
ACATACTACGCAGACTCCGTGAAGGGCCGGTTCACCATCTCCCGCGACAATTCCAAG
AACACGCTGTATCTGCAAATGAACAGCCTGCGTGCCGAGGACACTGCGGTATATTACT
GTGCGAAATGGTTTTATCGGAATTTTGACTACTGGGGTCAGGGAACCCTGGTCACCGT
CTCGAGC
DOM7r-36-103
(SEQ ID NO: 35)
GAGGTGCAGCTGTTGGAGTCTGGGGGAGGCTTGGTACAGCCTGGGGGGTCCCTGCG
TCTCTCCTGTGCAGCCTCCGGATTCACCTTTAATCATTATACGATGGGGTGGGTCCGC
CAGGCTCCAGGGAAGGGTCGAGAGTGGGTCTCATTGATTCATCCGAGTGGTACGGTG
ACATACTACGCAGACTCCGTGAAGGGCCGGTTCACCACCTCCCGCGACAATTCCAAG
AACACGCTGTATCTGCAAATGAACAGCCTGCGTGCCGAGGACACCGCGGTATATTACT
GTGCGAAATGGGATCGTAAGTTGTTTGACCACTGGGGTCAGGGAACCCTGGTCACCG
TCTCGAGC
DOM7r-36-104
(SEQ ID NO: 36)
GAGGTGCAGCTGTTGGAGTCTGGGGGAGGCTTGGTACAGCCTGGGGGGTCCCTGCG
TCTCTCCTGTGCAGCCTCCGGATTCACCTTTAATCATTATACGATGGGGTGGGTCCGC
CAGGCTCCAGGGAAGGGTCGAGAGTGGGTCTCATTGATTCATCCGAGTGGTACGGTG
ACATACTACGCAGACTCCGTGAAGGGCCGGTTCACCATCTCCCGCGACAATTCCAAG
AACACGCTGTATCTGCAAATGAACAGCCTGCGTGCCGAGGACACTGCGGTATATTACT
GTGCGAAATGGGCTACTAGGGGTTTTGACTACTGGGGTCAGGGAACCCTGGTCACCG
TCTCGAGC
DOM7r-36-105
(SEQ ID NO: 37)
GAGGTGCAGCTGTTGGAGTCTGGGGGAGGCTTGGTACAGCCTGGGGGGTCCCTGCG
TCTCTCCTGTGCAGCCTCCGGATTCACCTTTAATCATTATACGATGGGGTGGGTCCGC
CAGGCTCCAGGGAAGGGTCGAGAGTGGGTCTCATTGATTCATCCGAGTGGTACGGTG
ACATACTACGCAGACTCCGTGAAGGGCCGGTTCACCATCTCCCGCGACAATTCCAAG
AACACGCTGTATCTGCAAATGAACAGCCTGCGTGCCGAGGACACCGCGGTATATTACT
GTGCGAAATTGGGTACTAAGGTTTTTGACTACTGGGGTCAGGGAACCCTGGTCACCG
TCTCGAGC
DOM7r-36-106
(SEQ ID NO: 38)
GAGGTGCAGCTGTTGGAGTCTGGGGGAGGCTTGGTACAGCCTGGGGGGTCCCTGCG
TCTCTCCTGTGCAGCCTCCGGATTCACCTTTAATCATTATACGATGGGGTGGGTCCGC
CAGGCTCCAGGGAAGGGTCGAGAGTGGGTCTCATTGATTCATCCGAGTGGTACGGTG
ACATACTACGCAGACTCCGTGAAGGGCCGGTTCACCATCTCCCGCGACAATTCCAAG
AACACGCTGTATCTGCAAATGAACAGCCTGCGTGCCGAGGACACCGCGGTATATTACT
GTGCGAAATTTAGGGGGCGGGGTTTTGACTACTGGGGTCAGGGAACCCTGGTCACCG
TCTCGAGC
DOM7r-36-107
(SEQ ID NO: 39)
GAGGTGCAGCTGTTGGAGTCTGGGGGAGGCTTGGTACAGCCTGGGGGGTCCCTGCG
TCTCTCCTGTGCAGCCTCCGGATTCACCTTTAATCATTATACGATGGGGTGGGTCCGC
CAGGCTCCAGGGAAGGGTCGAGAGTGGGTCTCATTGATTCATCCGAGTGGTACGGTG
ACATACTACGCAGACTCCGTGAAGGGCCGGTTCACCATCTCCCGCGACAATTCCAAG
AACACGCTGTATCTGCAAATGAACAGCCTGCGTGCCGAGGACACCGCGGTATATTACT
GTGCGAAATGGGAGAAGAAGTATTTTGACTACTGGGGTCAGGGAACCCTGGTCACCG
TCTCGAGC
DOM7r-36-108
(SEQ ID NO: 40)
GAGGTGCAGCTGTTGGAGTCTGGGGGAGGCTTGGTACAGCCTGGGGGGTCCCTGCG
TCTCTCCTGTGCGGCCTCCGGATTCACCTTTAATCATTATACGATGGGGTGGGTCCGC
CAGGCTCCAGGGAAGGGTCGAGAGTGGGTCTCATTGATTCATCCGAGTGGTACGGTG
ACATACTACGCAGACTCCGTGAAGGGCCGGTTCACCACCTCCCGCGACAATTCCAAG
AACACGCTGTATCTGCAAATGAACAGCCTGCGTGCCGAGGACACCGCGGTATATTACT
GTGCGAAACTTGAGGGGCGGTCGTTTGACTACTGGGGTCAGGGAACCCTGGTCACC
GTCTCGAGC
DOM7r-92-4
(SEQ ID NO: 41)
GAGGTGCAGCTGTTGGAGTCTGGGGGAGGCTTGGTACAGCCTGGGGGGTCCCTGCG
TCTCTCCTGTGCAGCCTCCGGATTCACCTTTGATACGAGTAGTATGTTGTGGGTCCGC
CAGGCTCCAGGGAAGGGTCTAGAGTGGGTCTCAGTTATTCATCAGAGTGGTACGCCT
ACATACTACGCAGACTCCGTGAAGGGCCGGTTCACCATCTCCCGCGACAATTCCAAG
AACACGCTGTATCTGCAAATGAACAGCCTGCGTGCCGAGGACACCGCGGTATATTACT
GTGCGAAATTTCCGTCTACTCATGGTAAGTTTGACTACTGGGGTCAGGGAACCCTGGT
CACCGTCTCGAGC
DOM7r-92-100
(SEQ ID NO: 42)
GAGGTGCAGCTGTTGGAGTCTGGGGGAGGCTTGGTACAGCCTGGGGGGTCCCTGCG
TCTCTCCTGTGCAGCCTCCGGATTCACCTTTGATACGAGTAGTATGTTGTGGGTCCGC
CAGGCTCCAGGGAAGGGTCTAGAGTGGGTCTCAGTTATTCATCAGAGTGGTACGCCT
ACATACTACGCAGACTCCGTGAAGGGCCGGTTCACCATCTCCCGCGACAATTCCAAG
AACACGCTGTATCTGCAAATGAACAGCCTGCGTGCCGAGGACACCGCGGTATATTACT
GTGCGAAATTTCCGTCTTCTAGGATGAAGTTTGACTACTGGGGTCAGGGAACCCTGGT
CACCGTCTCGAGC
DOM7r-92-101
(SEQ ID NO: 43)
GAGGTGCAGCTGTTGGAGTCTGGGGGAGGCTTGGTACAGCCTGGGGGGTCCCTGCG
TCTCTCCTGTGCAGCCTCCGGATTCACCTTTGATACGAGTAGTATGTTGTGGGTCCGC
CAGGCTCCAGGGAAGGGTCTAGAGTGGGTCTCAGTTATTCATCAGAGTGGTACGCCT
ACATACTACGCAGACTCCGTGAAGGGCCGGTTCACCATCTCCCGCGACAATTCCAAG
AACACGCTGTATCTGCAAATGAACAGCCTGCGTGCCGAGGACACTGCGGTATATTACT
GTGCGAAATTTCCGTCTAGGAAGATGAAGTTTGACTACCGGGGTCAGGGAACCCTGG
TCACCGTCTCGAGC
DOM7r-92-102
(SEQ ID NO: 44)
GAGGTGCAGCTGTTGGAGTCTGGGGGAGGCTTGGTACAGCCTGGGGGGTCCCTGCG
TCTCTCCTGTGCAGCCTCCGGATTCACCTTTGATACGAGTAGTATGTTGTGGGTCCGC
CAGGCTCCAGGGAAGGGTCTAGAGTGGGTCTCAGTCATTCATCAGAGTGGTACGCCT
ACATACTACGCAGACTCCGTGAAGGGCCGGTTCACCATCTCCCGCGACAATTCCAAG
AACACGCTGTATCTGCAAATGAACAGCCTGCGTGCCGAGGACACCGCGGTATATTACT
GTGCGAAATTTCCGTCTTCTCAGTTTAGGTTTGACTACTGGGGTCAGGGAACCCTGGT
CACCGTCTCGAGC
DOM7r-92-103
(SEQ ID NO: 45)
GAGGTGCAGCTGTTGGAGTCTGGGGGAGGCTTGGTACAGCCTGGGGGGTCCCTGCG
TCTCTCCTGTGCAGCCTCCGGATTCACCTTTGATACGAGTAGTATGTTGTGGGTCCGC
CAGGCTCCAGGGAAGGGTCTAGAGTGGGTCTCAGTTATTCATCAGAGTGGTACGCCT
ACATACTACGCAGACTCCGTGAAGGGCCGGTTCACCATCTCCCGCGACAATTCCAAG
AACACGCTGTATCTGCAAATGAACAGCCTGCGTGCCGAGGACACTGCGGTATATTACT
GTGCGAAATTTCCGTCTGAGGGGATGAATTTTGACTACTGGGGTCAGGGAACCCTGG
TCACCGTCTCGAGC
DOM7r-31
(SEQ ID NO: 46)
GAGGTGCAGCTGTTGGAGTCTGGGGGAGGCTTGGTACAGCCTGGGGGGTCCCTGCG
TCTCTCCTGTACAGCCTCCGGATTCACCTTTAGGCATTATCGTATGGGTTGGGTCCGC
CAGGCTCCAGGGAAGGGTCTAGAGTGGGTCTCATGGATTCGTCCGGATGGTACGTTT
ACATACTACGCAGACTCCGTGAAGGGCCGGTTCACCATCTCCCGCGACAATTCCAAG
AACACGCTGTATCTGCAAATGAACAGCCTGCGTGCCGAGGACACCGCGGTATATTACT
GTGCGAAATCTTATATGGGTGATAGGTTTGACTACTGGGGTCAGGGAACCCTGGTCAC
CGTCTCGAGCG
DOM7r-36
(SEQ ID NO: 47)
GAGGTGCAGCTGTTGGAGTCTGGGGGAGGCTTGGTACAGCCTGGGGGGTCCCTGCG
TCTCTCCTGTGCAGCCTCCGGATTCACCTTTAATCATTATACGATGGGGTGGGTCCGC
CAGGCTCCAGGGAAGGGTCTAGAGTGGGTCTCATTGATTCATCCGAGTGGTACGGTG
ACATACTACGCAGACTCCGTGAAGGGCCGGTTCACCATCTCCCGCGACAATTCCAAG
AACACGCTGTATCTGCAAATGAACAGCCTGCGTGCCGAGGACACCGCGGTATATTACT
GTGCGAAATGGAGTTCGAGGGCGTTTGACTACTG
GGGTCAGGGAACCCTGGTCACCGTCTCGAGC
DOM7r-92
(SEQ ID NO: 48)
GAGGTGCAGCTGTTGGAGTCTGGGGGAGGCTTGGTACAGCCTGGGGGGTCCCTGCG
TCTCTCCTGTGCAGCCTCCGGATTCACCTTTGATACGAGTAGTATGTTGTGGGTCCGC
CAGGCTCCAGGGAAGGGTCTAGAGTGGGTCTCAGTTATTCATCAGAGTGGTACGCCT
iACATACTACGCAGACTCCGTGAAGGGCCGGTTCACCATCTCCCGCGACAATTCCAAG
AACACGCTGTATCTGCAAATGAACAGCCTGCGTGCCGAGGACACCGCGGTATATTACT
GTGCGAAATTTCCGTTTACTCATGGTAAGTTTGACTACTGGGGTCAGGGAACCCTGGT
CACCGTCTCGAGC
Note: All of the VH Albudabs and fusions used herein
have a myc tag

### EXEMPLIFICATION

Selection and affinity maturation of anti-SA immunoglobulin single variable domains is described for example in WO2008/096158, WO2010/094722 and WO2010/094723. PCT application, PCT/EP2010/06112, describes anti-SA immunoglobulin single variable domain parental sequences including a number of V_{H} sequences.

The parental sequences DOM7r31 (SEQ ID NO: 22), DOM7r36 (SEQ ID NO: 23) and DOM7r92 (SEQ ID NO: 24) were obtained from cross-over selections (human and rat SA) of VH naive libraries followed by screening against human and rat SA substantially as described above. Further selections were carried out as follows:

Individual error prone libraries (EP) of DOM7r-31 and DOM7r-36 were made. DOM7r-92 parental clone was pooled and combined in a single EP (error prone) library and screened together. All individual libraries were greater than 2×10⁹ PFU/mL.

Selections were performed in 4 rounds on soluble antigen (biotin-HSA; biotin-RSA; blocking with 2% Marvel) by cross over-selection with decreasing concentration of antigen: Round1 at 1 (HSA or RSA), Round 2 at 1 μM (RSA or HSA), followed by 2 further rounds of selection at 100 nM and 10 nM, respectively, with the same antigen as in Round 2. Ca. 3000 clones from both, R3 and R4 outputs were screened by supernatant BIAcore and clones ranked according to their off-rate (kd; sec⁻¹) only. Three lineages were selected based on their cross-species reactivity (binding to human, cyno, rat and mouse SA) and biochemical properties (in-solution state and thermal stability):

| Improved clone | From round |
|---|---|
| DOM7r-31-14 | R4 |
| DOM7r-36-2 | R4 |
| DOM7r-92-4 | R4 |

In a 2^{nd} affinity maturation, 14 NNK CDR-libraries (4-5 amino acids diversified per clone) were constructed based on the above three parental lineages with library sizes ranging from 1*10⁸ to 1*10⁹ PFU/mL. 4 rounds of selections were performed as described above using a cross-over selection approach against biotinylated, soluble antigen. The selections were divided into 2 groups:

Group 1: Round 1 at 1 μM human SA, Round 2 at 1 μM rat SA, Round 3 at 10 nM rat SA and Round 4 at 1 nM rat SA.

Group 2: Round 1 at 1 μM rat SA, Round 2 at 1 μM human SA, Round 3 at 10 nM human SA and Round 4 at 1 nM human SA.

BIAcore screening was performed using Round 3 and Round 4 selection outputs against human, rat, cyno and mouse SA. Clarified E. coli culture supernatants (OnEx expression for 48 hrs at 30C as described earlier/below) containing the expressed dAbs were used for the BIAcore screening and positive clones were selected based on their improved off-rate.

Unique dAbs were expressed as bacterial supernatants in 0.5 L shake flasks in Onex media at 30° C. for 48 hrs at 250 rpm. Cell cultures were clarified from bacterial cells by centrifugation. dAbs were purified from the clarified culture media by absorption to protein A streamline followed by elution with 100 mM glycine pH2.0.

To determine the binding affinity (K_{D}) of the AlbudAbs to Human, Rat, Mouse and Cynomolgus serum albumin, purified dAbs were analysed by BIAcore over a concentration range from 5000 nM to 39 nM (5000 nM, 2500 nM, 1250 nM, 312.5 nM, 156.25 nM, 78.125 nM, 39.0625 nM) using a CM5 Blacore chips covalently coated with various species' serum albumins.

MSA antigen was obtained from Sigma (essentially fatty acid free, ˜99% (agarose gel electrophoresis), lyophilized powder Cat. No. A3559) and CSA was purified from Cynomolgus serum albumin using prometic blue resin (Amersham).

### Biophysical Characterisation:

The routine bacterial expression level in 2.5 L shake flasks was determined spectrophotometrically following culture in Onex media at 30° C. for 48 hrs at 250 rpm.

The biophysical characteristics were determined by SEC MALLS and DSC.

SEC MALLS (size exclusion chromatography with multi-angle-LASER-light-scattering) is a non-invasive technique for the characterizing of macromolecules in solution. Briefly, proteins (at concentration of 1 mg/mL in buffer Dulbecco's PBS) are separated according to their hydrodynamic properties by size exclusion chromatography (column: TSK3000; S200). Following separation, the propensity of the protein to scatter light is measured using a multi-angle-LASER-light-scattering (MALLS) detector. The intensity of the scattered light while protein passes through the detector is measured as a function of angle. This measurement taken together with the protein concentration determined using an in-line refractive index (R1) detector allows calculation of the molar mass using appropriate equations (integral part of the analysis software Astra v.5.3.4.12). The highest concentration at the mid-point of the eluting peak is about 8-10 uM at an initial loading of a 1 mg/mL protein solution and this consequently is the concentration at which MALLS determines the in-solution state of the protein.

MALLS results: A single VH AlbudAb is 14 kDa in size. Any value between 14 and 28 kDa as determined by MALLS is indicative of varying degrees of self-association or dimer formation (i.e 16 kDa predominately monomeric under the conditions tested whereas 22 kDa indicates a strong propensity to dimerise under MALLS conditions).

DSC (Differential Scanning calorimetry): briefly, the protein is heated at a constant rate of 180 degrees C./hrs (at 1 mg/mL in PBS) and a detectable heat change associated with thermal denaturation measured. The transition midpoint (ₐₚₚTₘ) is determined, which is described as the temperature where 50% of the protein is in its native conformation and the other 50% is denatured. Here, DSC determined the apparent transition midpoint (appTm) as most of the proteins examined do not fully refold. The higher the Tm, the more stable the molecule. The software package used was Origin® v7.0383.

DSC results: The concentration of protein in a DSC experiment is much higher at 1 mg/mL in the actual reaction cell compared to MALLS. This higher concentration could explain in part the presence of two appTms for some AlbudAbs as seen in Table1; the first Tm constitutes the dissociation of the dimeric complex, whereas the second Tm represents the unfolding of the actual AlbudAb protein. Characteristics of the VH dAbs are summarised in Table 1 below.

TABLE 1

| | MALLS | | Expression |
|---|---|---|---|
| | (in-solution state: | DSC | level |
| | M = monomer; | (thermal unfolding | E. coli |
| | D = dimer | in ° C.) | (mg/L) |
| 7r31-14 | M | 67.0 (Tm1) 70.5 (Tm2) | n/a |
| (Parent) | | | |
| 7r31-100 | M | 64.5 (Tm1) 68.4 (Tm2) | 1.3 |
| 7r31-101 | M | 71.5 | 6.7 |
| 7r31-102 | M | 72.3 | 13.1 |
| 7r31-103 | M | 64.5 | 9.1 |
| 7r31-104 | M | 69.2 | 26.8 |
| 7r36-2 | M | 63.2 (Tm1) 68.7 (Tm2) | n/a |
| (Parent) | | | |
| 7r36-100 | M | 60.2 (Tm1) 65.7 (Tm2) | 8.1 |
| 7r36-101 | M | 56.4 (Tm1) 62.2 (Tm2) | 7.2 |
| 7r36-102 | M | n/d | 4 |
| 7r36-103 | M | 63.3 (Tm1) 67.0 (Tm2) | 0.8 |
| 7r36-104 | M | 70.7 | 18.4 |
| 7r36-105 | M | 66.8 | 5.3 |
| 7r36-106 | n/a | 68.5 | 54.6 |
| 7r36-107 | M | 66.6 | 49.4 |
| 7r36-108 | M | 66.2 (Tm1) 69.6 (Tm2) | 1.8 |
| 7r92-4 | M | 59.9 (Tm1) 61.5 (Tm2) | n/a |
| (Parent) | | | |
| 7r92-100 | Dimer 10%- | 57.2 | 45.3 |
| | Monomer 90% | | |
| 7r92-101 | M | 53.1 (Tm1) 59.2 (Tm2) | 10.3 |
| 7r92-102 | Trimer 10% & | 55.4 (Tm1) 59.0 (Tm2) | 8.4 |
| | Dimer/ | | |
| | Monomer 90% | | |
| 7r92-103 | M | 60.1 (Tm1) 62.9 (Tm2) | 20.1 |

n/a: not analysed

Cross-reactivity of the AlbudAbs™ (ie, anti-serum albumin dAbs) was determined against human, Cynomolgus monkey (cyno), rat and mouse serum albumin using surface plasmon resonance (SPR). In this case, Biacore™ was used. Biacore™ 2000/3000 was used for the supernatant screens and determination of binding kinetic all of dAbs described herein. Biacore™ T100 was used for the determination of cross-reactivity of lead Vk and VH Albudabs to a wider range of species' SAs (12 in total). Protein samples were run on the T100 BIAcore as described for the BIAcore2000/3000 experiments. Here, dilution series of purified proteins in BIAcore running buffer (HBS-EP) 1-in-2 dilution series from 5 uM down to 39 nM at a flow rate of 50 uL/min, contact time 60 secs, dissociation 200 sec. The BIAcore T100 is a technical development over the BIAcore 2000/3000 with higher sensitivity and improvement in the analysis software allowing for less subjective data analysis. Where appropriate, a kinetic Langmuir 1:1 fitting model was used. When very fast on- or off-rates did not allow a kinetic fitting routine, a steady state fit was used instead.

TABLE 2

| | BIAcore 3000 | | | |
|---|---|---|---|---|
| | KD [nM]; up to 4 independent measurements | | | |
| | performed on different protein batches | | | |
| | Human | Cynomolgus | Rat | Mouse |
| 7r31-14 | 208, 360, 1330, | n/a | 103, 90.2, 370 | 6, 12, 12, |
| (Parent) | 1950 | | | 14.2 |
| 7r31-100 | 260, 287 | n/a | 40, 40, 41 | 5.4, 12.7 |
| 7r31-101 | 367, 240 | n/a | 146, 30 | poor fit |
| 7r31-102 | 158, 100 | n/a | 67, 30 | poor fit |
| 7r31-103 | 200, 95 | n/a | 40 | poor fit |
| 7r31-104 | 89.1, 81 | 8560 | 11.9, 7.6 | poor fit |
| 7r36-2 | 190, 360, 305, | 800, 815, 628, | 467, 330, 244, | 520, 310, 1730 |
| (Parent) | 385 | 1500 | 229 | |
| 7r36-100 | 74, 100, 35 | 87, 230 | 105, 70 | 100 |
| 7r36-101 | 86 | 72 | 183 | n/a |
| 7r36-102 | 200, 54 | 430 | 100 | 250 |
| 7r36-103 | 62, 65 | 94 | 100, 57 | 270 |
| 7r36-104 | 82, 9 | 149, 360 | 109, 114 | 184, 313 |
| 7r36-105 | 284, 313 | 571, 1040 | 170 | 267, 1820 |
| 7r36-106 | 209, 124 | 840, 1100 | 75, 2, 42 | 113, 73 |
| 7r36-107 | 83.4, 44.5 | 127 | 97.3, 209 | 466 |
| 7r36-108 | 82, 67 | 265 | 76.4, 18 | 37.6 |
| 7r92-4 | 260, 420 | 90, 570 | 1000, 283 | 110, 860 |
| (Parent) | | | | |
| 7r92-100 | 1.4, 16, 7 | 5, 82 | 2.3, 115, 69 | 33, 90 |
| 7r92-101 | 15, 8 | 120 | 23, 16 | 56 |
| 7r92-102 | 28, 28 | 32 | 100 | 280 |
| 7r92-103 | 160, 123 | 770 | 3600 | 2000 |

n/a: not analysed as very low binding was evident
all proteins contained a myc tag.

TABLE 3
Cross-species reactivity for subset of VH AlbudAb leads as determined by
SPR using BIAcore T100. Values given are equilibrium binding (KD) in [M]

| | Human | Cynomolgus | Marmoset | Rat | Mouse | Dog |
|---|---|---|---|---|---|---|
| DOM7r-31- | 2.59E−08 | 3.13E−08 | 4.90E−06 | 6.09E−09 | 5.95E−08 | 8.26E−09 |
| 104 myc | | | | | | |
| DOM7r-36- | 1.83E−08 | 2.14E−08 | 1.11E−07 | 9.92E−08 | 1.43E−07 | 7.19E−07 |
| 107 myc | | | | | | |
| DOM7r-92- | 3.56E−09 | 1.07E−08 | 7.37E−09 | 5.43E−08 | 6.15E−08 | 4.50E−07 |
| 100 myc | | | | | | |

TABLE 4

| | Ferret | Pig | Mini Pig | Guinea Pig | Rabbit | Sheep |
|---|---|---|---|---|---|---|
| DOM7r-31- | 1.39E−08 | 4.15E−07 | 4.12E−07 | 1.63E−06 | 1.99E−06 | 2.91E−06 |
| 104 myc | | | | | | |
| DOM7r-36- | 7.40E−07 | 8.18E−07 | 8.95E−07 | NSB | NSB | 6.37E−07 |
| 107 myc | | | | | | |
| DOM7r-92- | 3.17E−07 | 1.91E−07 | 3.08E−07 | NSB | 1.36E−07 | 5.69E−07 |
| 100 myc | | | | | | |

NSB = no significant binding

### Example 2

### IFN-α2b-Vh Albudab rat PK studies

### Cloning and Expression

The affinity matured VH Albudabs were linked to Interferon alpha 2b (IFNα2b) to determine whether a useful PK of the AlbudAb was maintained as a fusion protein.

Interferon alpha 2b amino acid sequence:
(SEQ ID NO: 57)
CDLPQTHSLGSRRTLMLLAQMRRISLFSCLKDRHDFGFPQEEFGNQFQKAETIPVLHEMIQ
QIFNLFSTKDSSAAWDETLLDKFYTELYQQLNDLEACVIQGVGVTETPLMKEDSILAVRKYF
QRITLYLKEKKYSPCAWEVVRAEIMRSFSLSTNLQESLRSKE
Interferon alpha 2b nucleotide sequence:
(SEQ ID NO: 58)
TGTGATCTGCCTCAAACCCACAGCCTGGGTAGCAGGAGGACCTTGATGCTCCTGGCA
CAGATGAGGAGAATCTCTCTTTTCTCCTGCTTGAAGGACAGACATGACTTTGGATTTCC
CCAGGAGGAGTTTGGCAACCAGTTCCAAAAGGCTGAAACCATCCCTGTCCTCCATGA
GATGATCCAGCAGATCTTCAATCTCTTCAGCACAAAGGACTCATCTGCTGCTTGGGAT
GAGACCCTCCTAGACAAATTCTACACTGAACTCTACCAGCAGCTGAATGACCTGGAAG
CCTGTGTGATACAGGGGGTGGGGGTGACAGAGACTCCCCTGATGAAGGAGGACTCC
ATTCTGGCTGTGAGGAAATACTTCCAAAGAATCACTCTCTATCTGAAAGAGAAGAAATA
CAGCCCTTGTGCCTGGGAGGTTGTCAGAGCAGAAATCATGAGATCTTTTTCTTTGTCA
ACAAACTTGCAAGAAAGTTTAAGAAGTAAGGAA

IFNa2b was linked to the AlbudAb via a TVAAPS linker region (see WO2007085814). The constructs were cloned by SOE-PCR (single overlap extension according to the method of Horton et al. Gene, 77, p61 (1989)). PCR amplification of the AlbudAb and IFN sequences were carried out separately using primers with a ˜15 base pair overlap at the TVAAPS linker region.

IFNa2b SOE fragment
(SEQ ID NO: 59)
5′ GCCCGGATCCACCGGCTGTGATCTG
IFNa2b SOE fragment
(SEQ ID NO: 60)
3′ GGAGGATGGAGACTGGGTCATCTGGATGTC

The fragments were purified separately and subsequently assembled in a SOE (single overlap extension PCR extension) reaction using only the flanking primers.

IFNa2b SOE fragment
(SEQ ID NO: 61)
5′ GCCCGGATCCACCGGCTGTGATCTG

The assembled PCR product was digested using the restriction enzymes BamHI and HindIII and the gene ligated into the corresponding sites in the pDOM50, a mammalian expression vector which is a pTT5 derivative with an N-terminal V-J2-C mouse IgG secretory leader sequence to facilitate expression into the cell media.

Leader sequence (amino acid):
(SEQ ID NO: 62)
METDTLLLWVLLLWVPGSTG
Leader sequence (nucleotide):
(SEQ ID NO: 63)
ATGGAGACCGACACCCTGCTGCTGTGGGTGCTGCTGCTGTGGGTGCCCGG
ATCCACCGGGC

Plasmid DNA was prepared using QIAfilter megaprep (Qiagen). 1 μg DNA/ml was transfected with 293-Fectin into HEK293E cells and grown in serum free media. The protein is expressed in culture for 5 days and purified from culture supernatant using protein A affinity resin and eluted with 100 mM glycine pH2. The proteins were concentrated to greater than 1 mg/ml, buffer exchanged into PBS and endotoxin depleted using Q spin columns (Vivascience).

TABLE 5
Interferon alpha 2b-AlbudAb sequences with myc-tag
(as amino acid- and nucleotide sequence)
The Interferon alpha 2b is N-terminal to the
AlbudAb in the following fusions.

| | aa - myc | nt - myc |
|---|---|---|
| DMS7368 | CDLPQTHSLGSRRT | TGCGACTTGCCACAGACACATAGTTTG |
| (IFNa2b- | LMLLAQMRRISLFS | GGATCAAGAAGAACATTGATGTTATTAG |
| DOM7r-31-103 | CLKDRHDFGFPQEE | CACAAATGCGTAGAATTTCTTTGTTCTC |
| | FGNQFQKAETIPVL | TTGTCTAAAGGACCGTCACGACTTCGG |
| | HEMIQQIFNLFSTKD | ATTCCCTCAGGAAGAGTTTGGAAACCA |
| | SSAAWDETLLDKFY | ATTCCAAAAAGCAGAAACTATTCCTGTC |
| | TELYQQLNDLEACVI | TTGCACGAAATGATCCAGCAAATATTCA |
| | QGVGVTETPLMKED | ATTTGTTTTCTACAAAGGACTCATCAGC |
| | SILAVRKYFQRITLYL | CGCTTGGGATGAAACTCTGTTAGATAA |
| | KEKKYSPCAWEVV | ATTCTACACTGAACTATATCAACAACTG |
| | RAEIMRSFSLSTNL | AACGATCTAGAGGCTTGCGTTATTCAG |
| | QESLRSKETVAAPS | GGTGTAGGAGTTACTGAAACTCCCCTA |
| | EVQLLESGGGLVQP | ATGAAAGAAGATTCAATTCTAGCCGTTA |
| | GGSLRLSCTASGFT | GAAAATACTTTCAGCGTATCACATTGTA |
| | FRHYRMGWVRQAP | TTTAAAGGAAAAGAAATACTCCCCATGT |
| | GKGLEWVSWIRPD | GCATGGGAGGTGGTTAGAGCAGAAATT |
| | GTFTYYADSVKGRF | ATGAGGTCCTTCTCTCTTTCTACGAATT |
| | TISRDNSKNTLYLQ | TGCAAGAATCTTTGAGATCTAAGGAAA |
| | MNSLRAEDAAVYYC | CCGTCGCTGCTCCATCTGAGGTGCAGC |
| | AKSYMSGTFDYWG | TGTTGGAGTCTGGGGGAGGCTTGGTA |
| | QGTLVTVSS | CAGCCTGGGGGGTCCCTGCGTCTCTC |
| | (SEQID NO: 49) | CTGTACAGCCTCCGGATTCACCTTTAG |
| | | GCATTATCGTATGGGTTGGGTCCGCCA |
| | | GGCTCCAGGGAAGGGTCTAGAGTGGG |
| | | TCTCATGGATTCGTCCGGATGGTACGT |
| | | TTACATACTACGCAGACTCCGTGAAGG |
| | | GCCGGTTCACCATCTCCCGCGACAATT |
| | | CCAAGAACACGCTGTATCTGCAAATGA |
| | | ACAGCCTGCGTGCCGAGGACGCTGCG |
| | | GTATATTACTGTGCGAAATCTTATATGT |
| | | CTGGTACTTTTGACTACTGGGGTCAGG |
| | | GAACCCTGGTCACCGTCTCGAGC |
| | | (SEQ ID NO: 50) |
| DMS7369 | CDLPQTHSLGSRRT | TGCGACTTGCCACAGACACATAGTTTG |
| (IFNa2b- | LMLLAQMRRISLFS | GGATCAAGAAGAACATTGATGTTATTAG |
| DOM7r-36-100 | CLKDRHDFGFPQEE | CACAAATGCGTAGAATTTCTTTGTTCTC |
| | FGNQFQKAETIPVL | TTGTCTAAAGGACCGTCACGACTTCGG |
| | HEMIQQIFNLFSTKD | ATTCCCTCAGGAAGAGTTTGGAAACCA |
| | SSAAWDETLLDKFY | ATTCCAAAAAGCAGAAACTATTCCTGTC |
| | TELYQQLNDLEACVI | TTGCACGAAATGATCCAGCAAATATTCA |
| | QGVGVTETPLMKED | ATTTGTTTTCTACAAAGGACTCATCAGC |
| | SILAVRKYFQRITLYL | CGCTTGGGATGAAACTCTGTTAGATAA |
| | KEKKYSPCAWEVV | ATTCTACACTGAACTATATCAACAACTG |
| | RAEIMRSFSLSTNL | AACGATCTAGAGGCTTGCGTTATTCAG |
| | QESLRSKETVAAPS | GGTGTAGGAGTTACTGAAACTCCCCTA |
| | EVQLLESGGGLVQP | ATGAAAGAAGATTCAATTCTAGCCGTTA |
| | GGSLRLSCAASGFT | GAAAATACTTTCAGCGTATCACATTGTA |
| | FNHYTMGWVRQAP | TTTAAAGGAAAAGAAATACTCCCCATGT |
| | GKGREWVSLIHPSG | GCATGGGAGGTGGTTAGAGCAGAAATT |
| | TVTYYADSVKGRFTI | ATGAGGTCCTTCTCTCTTTCTACGAATT |
| | SRDNSKNTLYLQMN | TGCAAGAATCTTTGAGATCTAAGGAAA |
| | SLRAEDTAVYYCAK | CCGTCGCTGCTCCATCTGAGGTGCAGC |
| | WERRTFDYWGQGT | TGTTGGAGTCTGGGGGAGGCTTGGTA |
| | LVTVSS | CAGCCTGGGGGGTCCCTGCGTCTCTC |
| | (SEQ ID NO: 51) | CTGTGCAGCCTCCGGATTCACCTTTAA |
| | | TCATTATACGATGGGGTGGGTCCGCCA |
| | | GGCTCCAGGGAAGGGTCGAGAGTGGG |
| | | TCTCATTGATTCATCCGAGTGGTACGG |
| | | TGACATACTACGCAGACTCCGTGAAGG |
| | | GCCGGTTCACCATCTCCCGCGACAATT |
| | | CCAAGAACACGCTGTATCTGCAAATGA |
| | | ACAGCCTGCGTGCCGAGGACACCGCG |
| | | GTATATTACTGTGCGAAATGGGAGAGG |
| | | CGGACTTTTGACTACTGGGGTCAGGGA |
| | | ACCCTGGTCACCGTCTCGAGC |
| | | (SEQ ID NO: 52) |
| DMS7373 | CDLPQTHSLGSRRT | TGCGACTTGCCACAGACACATAGTTTG |
| (IFNa2b- | LMLLAQMRRISLFS | GGATCAAGAAGAACATTGATGTTATTAG |
| DOM7r-92-100 | CLKDRHDFGFPQEE | CACAAATGCGTAGAATTTCTTTGTTCTC |
| | FGNQFQKAETIPVL | TTGTCTAAAGGACCGTCACGACTTCGG |
| | HEMIQQIFNLFSTKD | ATTCCCTCAGGAAGAGTTTGGAAACCA |
| | SSAAWDETLLDKFY | ATTCCAAAAAGCAGAAACTATTCCTGTC |
| | TELYQQLNDLEACVI | TTGCACGAAATGATCCAGCAAATATTCA |
| | QGVGVTETPLMKED | ATTTGTTTTCTACAAAGGACTCATCAGC |
| | SILAVRKYFQRITLYL | CGCTTGGGATGAAACTCTGTTAGATAA |
| | KEKKYSPCAWEVV | ATTCTACACTGAACTATATCAACAACTG |
| | RAEIMRSFSLSTNL | AACGATCTAGAGGCTTGCGTTATTCAG |
| | QESLRSKETVAAPS | GGTGTAGGAGTTACTGAAACTCCCCTA |
| | EVQLLESGGGLVQP | ATGAAAGAAGATTCAATTCTAGCCGTTA |
| | GGSLRLSCAASGFT | GAAAATACTTTCAGCGTATCACATTGTA |
| | FDTSSMLWVRQAP | TTTAAAGGAAAAGAAATACTCCCCATGT |
| | GKGLEWVSVIHQSG | GCATGGGAGGTGGTTAGAGCAGAAATT |
| | TPTYYADSVKGRFTI | ATGAGGTCCTTCTCTCTTTCTACGAATT |
| | SRDNSKNTLYLQMN | TGCAAGAATCTTTGAGATCTAAGGAAA |
| | SLRAEDTAVYYCAK | CCGTCGCTGCTCCATCTGAGGTGCAGC |
| | FPSSRMKFDYWGQ | TGTTGGAGTCTGGGGGAGGCTTGGTA |
| | GTLVTVSS | CAGCCTGGGGGGTCCCTGCGTCTCTC |
| | (SEQ ID NO: 53) | CTGTGCAGCCTCCGGATTCACCTTTGA |
| | | TACGAGTAGTATGTTGTGGGTCCGCCA |
| | | GGCTCCAGGGAAGGGTCTAGAGTGGG |
| | | TCTCAGTTATTCATCAGAGTGGTACGC |
| | | CTACATACTACGCAGACTCCGTGAAGG |
| | | GCCGGTTCACCATCTCCCGCGACAATT |
| | | CCAAGAACACGCTGTATCTGCAAATGA |
| | | ACAGCCTGCGTGCCGAGGACACCGCG |
| | | GTATATTACTGTGCGAAATTTCCGTCTT |
| | | CTAGGATGAAGTTTGACTACTGGGGTC |
| | | AGGGAACCCTGGTCACCGTCTCGAGC |
| | | (SEQ ID NO: 54) |
| DMS7374 | CDLPQTHSLGSRRT | TGCGACTTGCCACAGACACATAGTTTG |
| (IFNa2b- | LMLLAQMRRISLFS | GGATCAAGAAGAACATTGATGTTATTAG |
| DOM7r-92-101 | CLKDRHDFGFPQEE | CACAAATGCGTAGAATTTCTTTGTTCTC |
| | FGNQFQKAETIPVL | TTGTCTAAAGGACCGTCACGACTTCGG |
| | HEMIQQIFNLFSTKD | ATTCCCTCAGGAAGAGTTTGGAAACCA |
| | SSAAWDETLLDKFY | ATTCCAAAAAGCAGAAACTATTCCTGTC |
| | TELYQQLNDLEACVI | TTGCACGAAATGATCCAGCAAATATTCA |
| | QGVGVTETPLMKED | ATTTGTTTTCTACAAAGGACTCATCAGC |
| | SILAVRKYFQRITLYL | CGCTTGGGATGAAACTCTGTTAGATAA |
| | KEKKYSPCAWEVV | ATTCTACACTGAACTATATCAACAACTG |
| | RAEIMRSFSLSTNL | AACGATCTAGAGGCTTGCGTTATTCAG |
| | QESLRSKETVAAPS | GGTGTAGGAGTTACTGAAACTCCCCTA |
| | EVQLLESGGGLVQP | ATGAAAGAAGATTCAATTCTAGCCGTTA |
| | GGSLRLSCAASGFT | GAAAATACTTTCAGCGTATCACATTGTA |
| | FDTSSMLWVRQAP | TTTAAAGGAAAAGAAATACTCCCCATGT |
| | GKGLEWVSVIHQSG | GCATGGGAGGTGGTTAGAGCAGAAATT |
| | TPTYYADSVKGRFTI | ATGAGGTCCTTCTCTCTTTCTACGAATT |
| | SRDNSKNTLYLQMN | TGCAAGAATCTTTGAGATCTAAGGAAA |
| | SLRAEDTAVYYCAK | CCGTCGCTGCTCCATCTGAGGTGCAGC |
| | FPSRKMKFDYRGQ | TGTTGGAGTCTGGGGGAGGCTTGGTA |
| | GTLVTVSS | CAGCCTGGGGGGTCCCTGCGTCTCTC |
| | (SEQ ID NO: 55) | CTGTGCAGCCTCCGGATTCACCTTTGA |
| | | TACGAGTAGTATGTTGTGGGTCCGCCA |
| | | GGCTCCAGGGAAGGGTCTAGAGTGGG |
| | | TCTCAGTTATTCATCAGAGTGGTACGC |
| | | CTACATACTACGCAGACTCCGTGAAGG |
| | | GCCGGTTCACCATCTCCCGCGACAATT |
| | | CCAAGAACACGCTGTATCTGCAAATGA |
| | | ACAGCCTGCGTGCCGAGGACACTGCG |
| | | GTATATTACTGTGCGAAATTTCCGTCTA |
| | | GGAAGATGAAGTTTGACTACCGGGGTC |
| | | AGGGAACCCTGGTCACCGTCTCGAGC |
| | | (SEQ ID NO: 56) |

### Determination of Serum Half Life in Rat

Interferon-AlbudAb fusions of DOM7r-31-103 myc; DOM7r-36-100 myc; DOM7r-92-100 myc; DOM7r-92-101 myc were cloned into the pDOM30 vector. For each AlbudAb, 20-50 mg quantities were expressed in HEK293 mammalian cells as described above and purified from clarified supernatant using protein A affinity resin and eluted with 100 mM glycine pH2. The proteins were concentrated to greater than 1 mg/mL, buffer exchanged into PBS and endotoxin depleted using Q spin columns (Vivascience). For Rat pharmacokinetic (PK) analysis, AlbudAbs were dosed as single i.v. injections at 2.0 mg/kg. Serum samples were taken at 0.16, 1, 4, 12, 24, 48, 72, 120, 168 hrs.

The procedure described is used to test animal samples from in vivo pharmacokinetic (PK) studies in order to determine the PK properties of Albudab-hIFNa2 molecules.

In this assay, a mouse anti-human IFNa2 monoclonal antibody (PBL Biomedical Laboratories, Cat No: 21105-1) is captured onto the surface of a 96-well standard bind MSD plate. This immobilised mAb is then used to capture the Albudab-humanIFNa2 in the serum sample. Bound AlbudAb-humanIFNa is detected using an appropriate sulfo-tagged detection antibody (rabbit anti-VH sulphotag; In-house supply Batch 090430DR-1). The signal from the assay is proportional to the amount of Albudab-humanIFNa2 in the sample and can be translated into actual ug/mL serum from a calibration curve. Rat serum was included at 10% for the determination of matrix fixing (Sera Labs, Cat No: S-909-D).

Results are shown in Table 6. All tested AlbudAbs show a serum-half life extending ability (negative control HEL4 dAb with T1/2 of 20 mins in rat) to varying degrees; this trend can also be seen in the calculated AUC being the highest value for the longest t1/2. The longest serum half-life with 40.6 hrs approximates the serum half-life of rat serum albumin.

Rat PK: hIFNa2b-DOM7r-31-103 myc; hIFNa2b-DOM7r-36-100 myc; hIFNa2b-DOM7r-92-100 myc; hIFNa2b-DOM7r-92-101 myc

TABLE 6
Rat PK parameters

| | KD(RSA) | T½ [hrs] | AUC 0-∞ | Clearance |
|---|---|---|---|---|
| | [nM] | in rat | [hr*ug/mL] | [mL/hr/kg] |
| IFN-a2b-L-DOM7r- | 66 | 26.6 | 2216 | 0.0009 |
| 31-103 myc | | | | |
| IFN-a2b-L-DOM7r- | 100 | 33.5 | 2126 | 0.0009 |
| 36-100 myc | | | | |
| IFN-a2b-L-DOM7r- | 50 | 37.9 | 1779 | 0.0011 |
| 92-100 myc | | | | |
| IFN-a2b-L-DOM7r- | 20 | 40.6 | 2464 | 0.0008 |
| 92-101 myc | | | | |

*The serum half-life of rat serum albumin is 35 hrs.
T½ is a measure of the circulation time of the molecule in the subjects.

As described for the Vk AlbudAb leads, an abbreviated rat PK feed and bleed study was conducted for IFN-a2b VH AlbudAb fusions. Upon fusion to therapeutic proteins, base Albudabs usually show a decrease in affinity to SA of about 2-10 fold. This explains the difference of affinity of the IFN-fusions compared to the corresponding base AlbudAbs (Table 6 above).
The IFN-Albudab fusions described here show the same direct correlation of KD vs. T1/2 (or affinity to serum albumin and serum residence time) as previously described (see WO/2010/094723; WO/2010/094722) for IFN-Vk AlbudAb fusions.

SEQUENCE LISTING TABLE

SEQ ID NO:

| Identifier | Amino acid | Nucleic acid |
|---|---|---|
| DOM7r-31-14 | 1 | 25 |
| DOM7r-31-100 | 2 | 26 |
| DOM7r-31-101 | 3 | 27 |
| DOM7r-31-102 | 4 | 28 |
| DOM7r-31-103 | 5 | 29 |
| DOM7r-31-104 | 6 | 30 |
| DOM7r-36-2 | 7 | 31 |
| DOM7r-36-100 | 8 | 32 |
| DOM7r-36-101 | 9 | 33 |
| DOM7r-36-102 | 10 | 34 |
| DOM7r-36-103 | 11 | 35 |
| DOM7r-36-104 | 12 | 36 |
| DOM7r-36-105 | 13 | 37 |
| DOM7r-36-106 | 14 | 38 |
| DOM7r-36-107 | 15 | 39 |
| DOM7r-36-108 | 16 | 40 |
| DOM7r-92-4 | 17 | 41 |
| DOM7r-92-100 | 18 | 42 |
| DOM7r-92-101 | 19 | 43 |
| DOM7r-92-102 | 20 | 44 |
| DOM7r-92-103 | 21 | 45 |
| DOM7r-31 | 22 | 46 |
| DOM7r-36 | 23 | 47 |
| DOM7r-92 | 24 | 48 |
| DMS7368 | 49 | 50 |
| (IFNα2b-DOM7r-31-103) | | |
| DMS7369 | 51 | 52 |
| (IFNα2b-DOM7r-36-100) | | |
| DMS7373 | 53 | 54 |
| (IFNα2b-DOM7r-92-100) | | |
| DMS7374 | 55 | 56 |
| (IFNα2b-DOM7r-92-101) | | |

1. An anti-serum albumin (SA) immunoglobulin single variable domain comprising an amino acid sequence that is at least 80% identical to an amino acid sequence selected from SEQ ID NOs: 1 to 21.

2. An anti-serum albumin (SA) immunoglobulin single variable domain comprising an amino acid sequence having up to 4 amino acid changes compared to an amino acid sequence selected from SEQ ID NOs: 1 to 21.

3. (canceled)

4. The variable domain of claim 1, wherein the variable domain comprises the amino acid sequence of any one of SEQ ID NOs: 1 to 21.

5. The variable domain of claim 1, comprising a binding site that specifically binds human SA with a dissociation constant (KD) of from about 0.1 to about 10000 nM, optionally from about 1 to about 6000 nM, as determined by surface plasmon resonance.

6. The variable domain of claim 1, comprising a binding site that specifically binds human SA with an off-rate constant (K_{d}) of from about 1.5×10⁻⁴ to about 0.1 sec⁻¹, optionally from about 3×10⁻⁴ to about 0.1 sec⁻¹ as determined by surface plasmon resonance.

7. The variable domain of claim 1, comprising a binding site that specifically binds human SA with an on-rate constant (Kₐ) of from about 2×10⁶ to about 1×10⁴M⁻¹ sec⁻¹, optionally from about 1×10⁶ to about 2×10⁴ M⁻¹sec⁻¹ as determined by surface plasmon resonance.

8. The variable domain of claim 1, comprising a binding site that specifically binds Cynomolgus monkey SA with a dissociation constant (KD) of from about 0.1 to about 10000 nM, optionally from about 1 to about 6000 nM, as determined by surface plasmon resonance.

9. The variable domain of claim 1, comprising a binding site that specifically binds Cynomolgus monkey SA with an off-rate constant (K_{d}) of from about 1.5×10⁻⁴ to about 0.1 sec⁻¹, optionally from about 3×10⁻⁴ to about 0.1 sec⁻¹ as determined by surface plasmon resonance.

10. The variable domain of claim 1, comprising a binding site that specifically binds Cynomolgus monkey SA with an on-rate constant (Kₐ) of from about 2×10⁶ to about 1×10⁴M⁻¹sec⁻¹, optionally from about 1×10⁶ to about 5×10³ M⁻¹sec⁻¹ as determined by surface plasmon resonance.

11. The variable domain of claim 1, wherein the variable domain has a melting temperature (Tm) of at least 55 degrees centigrade, optionally 55≦Tm≦75 degrees centigrade, as determined by DSC (differential scanning calorimetry).

12. The variable domain of claim 1, wherein the variable domain is substantially monomeric as determined by SEC-MALLS (size exclusion chromatography with multi-angle-LASER-light-scattering).

13. A multispecific ligand comprising an anti-SA variable domain of claim 1 and a binding moiety that specifically binds a target antigen other than SA, optionally wherein the binding moiety is an TNFR1 antagonist.

14. The anti-SA single variable domain of claim 1, wherein the variable domain is conjugated to an NCE drug.

15. A fusion protein comprising a polypeptide or peptide drug fused to a variable domain according to claim 1.

16. A composition comprising a variable domain, fusion protein or ligand of claim 1 and a pharmaceutically acceptable diluent, carrier, excipient or vehicle.

17. A nucleic acid comprising a nucleotide sequence encoding a variable domain according to claim 1.

18. A nucleic acid comprising a nucleotide sequence that is at least 80% identical to a sequence selected from SEQ ID NOs 25 to 45.

19. A vector comprising the nucleic acid of claim 17.

20. An isolated host cell comprising the vector of claim 19.

21. A method of treating or preventing a disease or disorder in a patient, comprising administering at least one dose of a variable domain according to claim 1.


SEQUENCE LISTING (63 sequences)

SEQ ID NO: 1 - protein, 118 aa, Artificial Sequence
  Amino acid sequence identified using molecular biology techniques.
EVQLLESGGGLVQPGGSLRLSCTASGFTFRHYRMGWVRQAPGKGLEWVSWIRPDGTFTYYADSVKGRFTISRDNSKNTLYLQMNSLRAEDTAVYYCAKSYMADRFDYWGQGTLVTVSS

SEQ ID NO: 2 - protein, 118 aa, Artificial Sequence
  Amino acid sequence identified using molecular biology techniques.
EVQLLEPGGGLVQPGGSLRLSCTASGFTFRHYRMGWVRQAPGKGLEWVSWIRPDGTFTYYADSVKGRFTISRDNSKNTLYLQMNSLRAEDTAVYYCAKSYLSGTFDYWGQGTLVTVSS

SEQ ID NO: 3 - protein, 118 aa, Artificial Sequence
  Amino acid sequence identified using molecular biology techniques.
EVQLLESGGGLVQPGGSLRLSCTASGFTFRHYRMGWVRQAPGKGLEWVSWIRPDGTFTYYADSVKGRFTISRDNSKNTLYLQMNSLRAEDTAVYYCAKSYQAGTFDYWGQGTLVTVSS

SEQ ID NO: 4 - protein, 118 aa, Artificial Sequence
  Amino acid sequence identified using molecular biology techniques.
EVQLLESGGGLVQPGGSLRLSCTASGFTFRHYRMGWVRQAPGKGLEWVSWIRPDGTFTYYADSVKGRFTISRDNSKNTLYLQMNSLRAEDTAVYYCAKSYHADKFDYWGQGTLVTVSS

SEQ ID NO: 5 - protein, 118 aa, Artificial Sequence
  Amino acid sequence identified using molecular biology techniques.
EVQLLESGGGLVQPGGSLRLSCTASGFTFRHYRMGWVRQAPGKGLEWVSWIRPDGTFTYYADSVKGRFTISRDNSKNTLYLQMNSLRAEDAAVYYCAKSYMSGTFDYWGQGTLVTVSS

SEQ ID NO: 6 - protein, 118 aa, Artificial Sequence
  Amino acid sequence identified using molecular biology techniques.
EVQLLESGGGLVQPGGSLRLSCTASGFTFRHYRMGWVRQAPGKGLEWVSWIRPDGTFTYYADSVKGRFTISRDNSKNTLYLQMNSLRAEDTAVYYCAKSYMAGTFDYWGQGTLVTVSS

SEQ ID NO: 7 - protein, 117 aa, Artificial Sequence
  Amino acid sequence identified using molecular biology techniques.
EVQLLESGGGLVQPGGSLRLSCAASGFTFNHYTMGWVRQAPGKGREWVSLIHPSGTVTYYADSVKGRFTISRDNSKNTLYLQMNSLRAEDTAVYYCAKWSSRAFDYWGQGTLVTVSS

SEQ ID NO: 8 - protein, 117 aa, Artificial Sequence
  Amino acid sequence identified using molecular biology techniques.
EVQLLESGGGLVQPGGSLRLSCAASGFTFNHYTMGWVRQAPGKGREWVSLIHPSGTVTYYADSVKGRFTISRDNSKNTLYLQMNSLRAEDTAVYYCAKWERRTFDYWGQGTLVTVSS

SEQ ID NO: 9 - protein, 117 aa, Artificial Sequence
  Amino acid sequence identified using molecular biology techniques.
EVQLLESGGGLVQPGGSLRLSCAASGFTFNHYTMGWVRQAPGKGREWVSLIHPSGTVTYYADSVKGRFTISRDNSKNTLYLQMNSLRAEDTAVYYCAKWFLKSFDYWGQGTLVTVSS

SEQ ID NO: 10 - protein, 117 aa, Artificial Sequence
  Amino acid sequence identified using molecular biology techniques.
EVQLLESGGGLVQPGGSLRLSCAASGFTFNHYTMGWVRQAPGKGREWVSLIHPSGTVTYYADSVKGRFTISRDNSKNTLYLQMNSLRAEDTAVYYCAKWFYRNFDYWGQGTLVTVSS

SEQ ID NO: 11 - protein, 117 aa, Artificial Sequence
  Amino acid sequence identified using molecular biology techniques.
EVQLLESGGGLVQPGGSLRLSCAASGFTFNHYTMGWVRQAPGKGREWVSLIHPSGTVTYYADSVKGRFTTSRDNSKNTLYLQMNSLRAEDTAVYYCAKWDRKLFDHWGQGTLVTVSS

SEQ ID NO: 12 - protein, 117 aa, Artificial Sequence
  Amino acid sequence identified using molecular biology techniques.
EVQLLESGGGLVQPGGSLRLSCAASGFTFNHYTMGWVRQAPGKGREWVSLIHPSGTVTYYADSVKGRFTISRDNSKNTLYLQMNSLRAEDTAVYYCAKWATRGFDYWGQGTLVTVSS

SEQ ID NO: 13 - protein, 117 aa, Artificial Sequence
  Amino acid sequence identified using molecular biology techniques.
EVQLLESGGGLVQPGGSLRLSCAASGFTFNHYTMGWVRQAPGKGREWVSLIHPSGTVTYYADSVKGRFTISRDNSKNTLYLQMNSLRAEDTAVYYCAKLGTKVFDYWGQGTLVTVSS

SEQ ID NO: 14 - protein, 117 aa, Artificial Sequence
  Amino acid sequence identified using molecular biology techniques.
EVQLLESGGGLVQPGGSLRLSCAASGFTFNHYTMGWVRQAPGKGREWVSLIHPSGTVTYYADSVKGRFTISRDNSKNTLYLQMNSLRAEDTAVYYCAKFRGRGFDYWGQGTLVTVSS

SEQ ID NO: 15 - protein, 117 aa, Artificial Sequence
  Amino acid sequence identified using molecular biology techniques.
EVQLLESGGGLVQPGGSLRLSCAASGFTFNHYTMGWVRQAPGKGREWVSLIHPSGTVTYYADSVKGRFTISRDNSKNTLYLQMNSLRAEDTAVYYCAKWEKKYFDYWGQGTLVTVSS

SEQ ID NO: 16 - protein, 117 aa, Artificial Sequence
  Amino acid sequence identified using molecular biology techniques.
EVQLLESGGGLVQPGGSLRLSCAASGFTFNHYTMGWVRQAPGKGREWVSLIHPSGTVTYYADSVKGRFTTSRDNSKNTLYLQMNSLRAEDTAVYYCAKLEGRSFDYWGQGTLVTVSS

SEQ ID NO: 17 - protein, 119 aa, Artificial Sequence
  Amino acid sequence identified using molecular biology techniques.
EVQLLESGGGLVQPGGSLRLSCAASGFTFDTSSMLWVRQAPGKGLEWVSVIHQSGTPTYYADSVKGRFTISRDNSKNTLYLQMNSLRAEDTAVYYCAKFPSTHGKFDYWGQGTLVTVSS

SEQ ID NO: 18 - protein, 119 aa, Artificial Sequence
  Amino acid sequence identified using molecular biology techniques.
EVQLLESGGGLVQPGGSLRLSCAASGFTFDTSSMLWVRQAPGKGLEWVSVIHQSGTPTYYADSVKGRFTISRDNSKNTLYLQMNSLRAEDTAVYYCAKFPSSRMKFDYWGQGTLVTVSS

SEQ ID NO: 19 - protein, 119 aa, Artificial Sequence
  Amino acid sequence identified using molecular biology techniques.
EVQLLESGGGLVQPGGSLRLSCAASGFTFDTSSMLWVRQAPGKGLEWVSVIHQSGTPTYYADSVKGRFTISRDNSKNTLYLQMNSLRAEDTAVYYCAKFPSRKMKFDYRGQGTLVTVSS

SEQ ID NO: 20 - protein, 119 aa, Artificial Sequence
  Amino acid sequence identified using molecular biology techniques.
EVQLLESGGGLVQPGGSLRLSCAASGFTFDTSSMLWVRQAPGKGLEWVSVIHQSGTPTYYADSVKGRFTISRDNSKNTLYLQMNSLRAEDTAVYYCAKFPSSQFRFDYWGQGTLVTVSS

SEQ ID NO: 21 - protein, 119 aa, Artificial Sequence
  Amino acid sequence identified using molecular biology techniques.
EVQLLESGGGLVQPGGSLRLSCAASGFTFDTSSMLWVRQAPGKGLEWVSVIHQSGTPTYYADSVKGRFTISRDNSKNTLYLQMNSLRAEDTAVYYCAKFPSEGMNFDYWGQGTLVTVSS

SEQ ID NO: 22 - protein, 118 aa, Artificial Sequence
  Amino acid sequence identified using molecular biology techniques.
EVQLLESGGGLVQPGGSLRLSCTASGFTFRHYRMGWVRQAPGKGLEWVSWIRPDGTFTYYADSVKGRFTISRDNSKNTLYLQMNSLRAEDTAVYYCAKSYMGDRFDYWGQGTLVTVSS

SEQ ID NO: 23 - protein, 117 aa, Artificial Sequence
  Amino acid sequence identified using molecular biology techniques.
EVQLLESGGGLVQPGGSLRLSCAASGFTFNHYTMGWVRQAPGKGLEWVSLIHPSGTVTYYADSVKGRFTISRDNSKNTLYLQMNSLRAEDTAVYYCAKWSSRAFDYWGQGTLVTVSS

SEQ ID NO: 24 - protein, 119 aa, Artificial Sequence
  Amino acid sequence identified using molecular biology techniques.
EVQLLESGGGLVQPGGSLRLSCAASGFTFDTSSMLWVRQAPGKGLEWVSVIHQSGTPTYYADSVKGRFTISRDNSKNTLYLQMNSLRAEDTAVYYCAKFPFTHGKFDYWGQGTLVTVSS

SEQ ID NO: 25 - DNA, 354 bp, Artificial Sequence
  Nucleic acid sequence identified using molecular biology techniques.
gaggtgcagctgttggagtctgggggaggcttggtacagcctggggggtccctgcgtctctcctgtacagcctccggattcacctttaggcattatcgtatgggttgggtccgccaggctccagggaagggtctagagtgggtctcatggattcgtccggatggtacgtttacatactacgcagactccgtgaagggccggttcaccatctcccgcgacaattccaagaacacgctgtatctgcaaatgaacagcctgcgtgccgaggacaccgcggtatattactgtgcgaaatcttatatggctgataggtttgactactggggtcagggaaccctggtcaccgtctcgagc

SEQ ID NO: 26 - DNA, 354 bp, Artificial Sequence
  Nucleic acid sequence identified using molecular biology techniques.
gaggtgcagctgttggagcctgggggaggcttggtacagcctggggggtccctgcgtctctcctgtacagcctccggattcacctttaggcattatcgtatgggttgggtccgccaggctccagggaagggtctagagtgggtctcatggattcgtccggatggtacgtttacatactacgcagactccgtgaagggccggttcaccatctcccgcgacaattccaagaacacgctgtatctgcaaatgaacagcctgcgtgccgaggacaccgcggtatattactgtgcgaaatcttatcttagtggtacttttgactactggggtcagggaaccctggtcaccgtctcgagc

SEQ ID NO: 27 - DNA, 354 bp, Artificial Sequence
  Nucleic acid sequence identified using molecular biology techniques.
gaggtgcagctgttggagtctgggggaggcctggtacagcctggggggtccctgcgtctctcctgtacagcctccggattcacctttaggcattatcgtatgggttgggtccgccaggctccagggaagggtctagagtgggtctcatggattcgtccggatggtacgtttacatactacgcagactccgtgaagggccggttcaccatctcccgcgacaattccaagaacacgctgtatctgcaaatgaacagcctgcgtgccgaggacaccgcggtatattactgtgcgaaatcttatcaggctggtacgtttgactactggggtcagggaaccctggtcaccgtctcgagc

SEQ ID NO: 28 - DNA, 354 bp, Artificial Sequence
  Nucleic acid sequence identified using molecular biology techniques.
gaggtgcagctgttggagtctgggggaggcttggtacagcctggggggtccctgcgtctctcctgtacagcctccggattcacctttaggcattatcgtatgggttgggtccgccaggctccagggaagggtctagagtgggtctcatggattcgtccggatggtacgtttacatactacgcagactccgtgaagggccggttcaccatctcccgcgacaattccaagaacacgctgtatctgcaaatgaacagcctgcgtgccgaggacaccgcggtatattactgtgcgaaatcttatcatgcggataagtttgactactggggtcagggaaccctggtcaccgtctcgagc

SEQ ID NO: 29 - DNA, 354 bp, Artificial Sequence
  Nucleic acid sequence identified using molecular biology techniques.
gaggtgcagctgttggagtctgggggaggcttggtacagcctggggggtccctgcgtctctcctgtacagcctccggattcacctttaggcattatcgtatgggttgggtccgccaggctccagggaagggtctagagtgggtctcatggattcgtccggatggtacgtttacatactacgcagactccgtgaagggccggttcaccatctcccgcgacaattccaagaacacgctgtatctgcaaatgaacagcctgcgtgccgaggacgctgcggtatattactgtgcgaaatcttatatgtctggtacttttgactactggggtcagggaaccctggtcaccgtctcgagc

SEQ ID NO: 30 - DNA, 354 bp, Artificial Sequence
  Nucleic acid sequence identified using molecular biology techniques.
gaggtgcagctgttggagtctgggggaggcttggtacagcctggggggtccctgcgtctctcctgtacagcctccggattcacctttaggcattatcgtatgggttgggtccgccaggctccagggaagggtctagagtgggtctcatggattcgtccggatggtacgtttacatactacgcagactccgtgaagggccggttcaccatctcccgcgacaattccaagaacacgctgtatctgcaaatgaacagcctgcgtgccgaggacactgcggtatattactgtgcgaaatcttatatggctgggacgtttgactactggggtcagggaaccctggtcaccgtctcgagc

SEQ ID NO: 31 - DNA, 351 bp, Artificial Sequence
  Nucleic acid sequence identified using molecular biology techniques.
gaggtgcagctgttggagtctgggggaggcttggtacagcctggggggtccctgcgtctctcctgtgcagcctccggattcacctttaatcattatacgatggggtgggtccgccaggctccagggaagggtcgagagtgggtctcattgattcatccgagtggtacggtgacatactacgcagactccgtgaagggccggttcaccatctcccgcgacaattccaagaacacgctgtatctgcaaatgaacagcctgcgtgccgaggacaccgcggtatattactgtgcgaaatggagttcgagggcgtttgactactggggtcagggaaccctggtcaccgtctcgagc

SEQ ID NO: 32 - DNA, 351 bp, Artificial Sequence
  Nucleic acid sequence identified using molecular biology techniques.
gaggtgcagctgttggagtctgggggaggcttggtacagcctggggggtccctgcgtctctcctgtgcagcctccggattcacctttaatcattatacgatggggtgggtccgccaggctccagggaagggtcgagagtgggtctcattgattcatccgagtggtacggtgacatactacgcagactccgtgaagggccggttcaccatctcccgcgacaattccaagaacacgctgtatctgcaaatgaacagcctgcgtgccgaggacaccgcggtatattactgtgcgaaatgggagaggcggacttttgactactggggtcagggaaccctggtcaccgtctcgagc

SEQ ID NO: 33 - DNA, 351 bp, Artificial Sequence
  Nucleic acid sequence identified using molecular biology techniques.
gaggtgcagctgttggagtctgggggaggcttggtacagcctggggggtccctgcgtctctcctgtgcagcctccggattcacctttaatcattatacgatggggtgggtccgccaggctccagggaagggtcgagagtgggtctcattgattcatccgagtggtacggtgacatactacgcagactccgtgaagggccggttcaccatctcccgcgacaattccaagaacacgctgtatctgcaaatgaacagcctgcgtgccgaggacaccgcggtatattactgtgcgaaatggtttctgaagagttttgactactggggtcagggaaccctggtcaccgtctcgagc

SEQ ID NO: 34 - DNA, 351 bp, Artificial Sequence
  Nucleic acid sequence identified using molecular biology techniques.
gaggtgcagctgttggagtctgggggaggcttggtacagcctggggggtccctgcgtctctcctgtgcagcctccggattcacctttaatcattatacgatggggtgggtccgccaggctccagggaagggtcgagagtgggtctcattgattcacccgagtggtacggtgacatactacgcagactccgtgaagggccggttcaccatctcccgcgacaattccaagaacacgctgtatctgcaaatgaacagcctgcgtgccgaggacactgcggtatattactgtgcgaaatggttttatcggaattttgactactggggtcagggaaccctggtcaccgtctcgagc

SEQ ID NO: 35 - DNA, 351 bp, Artificial Sequence
  Nucleic acid sequence identified using molecular biology techniques.
gaggtgcagctgttggagtctgggggaggcttggtacagcctggggggtccctgcgtctctcctgtgcagcctccggattcacctttaatcattatacgatggggtgggtccgccaggctccagggaagggtcgagagtgggtctcattgattcatccgagtggtacggtgacatactacgcagactccgtgaagggccggttcaccacctcccgcgacaattccaagaacacgctgtatctgcaaatgaacagcctgcgtgccgaggacaccgcggtatattactgtgcgaaatgggatcgtaagttgtttgaccactggggtcagggaaccctggtcaccgtctcgagc

SEQ ID NO: 36 - DNA, 351 bp, Artificial Sequence
  Nucleic acid sequence identified using molecular biology techniques.
gaggtgcagctgttggagtctgggggaggcttggtacagcctggggggtccctgcgtctctcctgtgcagcctccggattcacctttaatcattatacgatggggtgggtccgccaggctccagggaagggtcgagagtgggtctcattgattcatccgagtggtacggtgacatactacgcagactccgtgaagggccggttcaccatctcccgcgacaattccaagaacacgctgtatctgcaaatgaacagcctgcgtgccgaggacactgcggtatattactgtgcgaaatgggctactaggggttttgactactggggtcagggaaccctggtcaccgtctcgagc

SEQ ID NO: 37 - DNA, 351 bp, Artificial Sequence
  Nucleic acid sequence identified using molecular biology techniques.
gaggtgcagctgttggagtctgggggaggcttggtacagcctggggggtccctgcgtctctcctgtgcagcctccggattcacctttaatcattatacgatggggtgggtccgccaggctccagggaagggtcgagagtgggtctcattgattcatccgagtggtacggtgacatactacgcagactccgtgaagggccggttcaccatctcccgcgacaattccaagaacacgctgtatctgcaaatgaacagcctgcgtgccgaggacaccgcggtatattactgtgcgaaattgggtactaaggtttttgactactggggtcagggaaccctggtcaccgtctcgagc

SEQ ID NO: 38 - DNA, 351 bp, Artificial Sequence
  Nucleic acid sequence identified using molecular biology techniques.
gaggtgcagctgttggagtctgggggaggcttggtacagcctggggggtccctgcgtctctcctgtgcagcctccggattcacctttaatcattatacgatggggtgggtccgccaggctccagggaagggtcgagagtgggtctcattgattcatccgagtggtacggtgacatactacgcagactccgtgaagggccggttcaccatctcccgcgacaattccaagaacacgctgtatctgcaaatgaacagcctgcgtgccgaggacaccgcggtatattactgtgcgaaatttagggggcggggttttgactactggggtcagggaaccctggtcaccgtctcgagc

SEQ ID NO: 39 - protein, 351 aa, Artificial Sequence
  Amino acid sequence identified using molecular biology techniques.
GAGGTGCAGCTGTTGGAGTCTGGGGGAGGCTTGGTACAGCCTGGGGGGTCCCTGCGTCTCTCCTGTGCAGCCTCCGGATTCACCTTTAATCATTATACGATGGGGTGGGTCCGCCAGGCTCCAGGGAAGGGTCGAGAGTGGGTCTCATTGATTCATCCGAGTGGTACGGTGACATACTACGCAGACTCCGTGAAGGGCCGGTTCACCATCTCCCGCGACAATTCCAAGAACACGCTGTATCTGCAAATGAACAGCCTGCGTGCCGAGGACACCGCGGTATATTACTGTGCGAAATGGGAGAAGAAGTATTTTGACTACTGGGGTCAGGGAACCCTGGTCACCGTCTCGAGC

SEQ ID NO: 40 - DNA, 351 bp, Artificial Sequence
  Nucleic acid sequence identified using molecular biology techniques.
gaggtgcagctgttggagtctgggggaggcttggtacagcctggggggtccctgcgtctctcctgtgcggcctccggattcacctttaatcattatacgatggggtgggtccgccaggctccagggaagggtcgagagtgggtctcattgattcatccgagtggtacggtgacatactacgcagactccgtgaagggccggttcaccacctcccgcgacaattccaagaacacgctgtatctgcaaatgaacagcctgcgtgccgaggacaccgcggtatattactgtgcgaaacttgaggggcggtcgtttgactactggggtcagggaaccctggtcaccgtctcgagc

SEQ ID NO: 41 - DNA, 357 bp, Artificial Sequence
  Nucleic acid sequence identified using molecular biology techniques.
gaggtgcagctgttggagtctgggggaggcttggtacagcctggggggtccctgcgtctctcctgtgcagcctccggattcacctttgatacgagtagtatgttgtgggtccgccaggctccagggaagggtctagagtgggtctcagttattcatcagagtggtacgcctacatactacgcagactccgtgaagggccggttcaccatctcccgcgacaattccaagaacacgctgtatctgcaaatgaacagcctgcgtgccgaggacaccgcggtatattactgtgcgaaatttccgtctactcatggtaagtttgactactggggtcagggaaccctggtcaccgtctcgagc

SEQ ID NO: 42 - DNA, 357 bp, Artificial Sequence
  Nucleic acid sequence identified using molecular biology techniques.
gaggtgcagctgttggagtctgggggaggcttggtacagcctggggggtccctgcgtctctcctgtgcagcctccggattcacctttgatacgagtagtatgttgtgggtccgccaggctccagggaagggtctagagtgggtctcagttattcatcagagtggtacgcctacatactacgcagactccgtgaagggccggttcaccatctcccgcgacaattccaagaacacgctgtatctgcaaatgaacagcctgcgtgccgaggacaccgcggtatattactgtgcgaaatttccgtcttctaggatgaagtttgactactggggtcagggaaccctggtcaccgtctcgagc

SEQ ID NO: 43 - DNA, 357 bp, Artificial Sequence
  Nucleic acid sequence identified using molecular biology techniques.
gaggtgcagctgttggagtctgggggaggcttggtacagcctggggggtccctgcgtctctcctgtgcagcctccggattcacctttgatacgagtagtatgttgtgggtccgccaggctccagggaagggtctagagtgggtctcagttattcatcagagtggtacgcctacatactacgcagactccgtgaagggccggttcaccatctcccgcgacaattccaagaacacgctgtatctgcaaatgaacagcctgcgtgccgaggacactgcggtatattactgtgcgaaatttccgtctaggaagatgaagtttgactaccggggtcagggaaccctggtcaccgtctcgagc

SEQ ID NO: 44 - DNA, 357 bp, Artificial Sequence
  Nucleic acid sequence identified using molecular biology techniques.
gaggtgcagctgttggagtctgggggaggcttggtacagcctggggggtccctgcgtctctcctgtgcagcctccggattcacctttgatacgagtagtatgttgtgggtccgccaggctccagggaagggtctagagtgggtctcagtcattcatcagagtggtacgcctacatactacgcagactccgtgaagggccggttcaccatctcccgcgacaattccaagaacacgctgtatctgcaaatgaacagcctgcgtgccgaggacaccgcggtatattactgtgcgaaatttccgtcttctcagtttaggtttgactactggggtcagggaaccctggtcaccgtctcgagc

SEQ ID NO: 45 - DNA, 357 bp, Artificial Sequence
  Nucleic acid sequence identified using molecular biology techniques.
gaggtgcagctgttggagtctgggggaggcttggtacagcctggggggtccctgcgtctctcctgtgcagcctccggattcacctttgatacgagtagtatgttgtgggtccgccaggctccagggaagggtctagagtgggtctcagttattcatcagagtggtacgcctacatactacgcagactccgtgaagggccggttcaccatctcccgcgacaattccaagaacacgctgtatctgcaaatgaacagcctgcgtgccgaggacactgcggtatattactgtgcgaaatttccgtctgaggggatgaattttgactactggggtcagggaaccctggtcaccgtctcgagc

SEQ ID NO: 46 - DNA, 355 bp, Artificial Sequence
  Nucleic acid sequence identified using molecular biology techniques.
gaggtgcagctgttggagtctgggggaggcttggtacagcctggggggtccctgcgtctctcctgtacagcctccggattcacctttaggcattatcgtatgggttgggtccgccaggctccagggaagggtctagagtgggtctcatggattcgtccggatggtacgtttacatactacgcagactccgtgaagggccggttcaccatctcccgcgacaattccaagaacacgctgtatctgcaaatgaacagcctgcgtgccgaggacaccgcggtatattactgtgcgaaatcttatatgggtgataggtttgactactggggtcagggaaccctggtcaccgtctcgagcg

SEQ ID NO: 47 - DNA, 351 bp, Artificial Sequence
  Nucleic acid sequence identified using molecular biology techniques.
gaggtgcagctgttggagtctgggggaggcttggtacagcctggggggtccctgcgtctctcctgtgcagcctccggattcacctttaatcattatacgatggggtgggtccgccaggctccagggaagggtctagagtgggtctcattgattcatccgagtggtacggtgacatactacgcagactccgtgaagggccggttcaccatctcccgcgacaattccaagaacacgctgtatctgcaaatgaacagcctgcgtgccgaggacaccgcggtatattactgtgcgaaatggagttcgagggcgtttgactactggggtcagggaaccctggtcaccgtctcgagc

SEQ ID NO: 48 - DNA, 357 bp, Artificial Sequence
  Nucleic acid sequence identified using molecular biology techniques.
gaggtgcagctgttggagtctgggggaggcttggtacagcctggggggtccctgcgtctctcctgtgcagcctccggattcacctttgatacgagtagtatgttgtgggtccgccaggctccagggaagggtctagagtgggtctcagttattcatcagagtggtacgcctacatactacgcagactccgtgaagggccggttcaccatctcccgcgacaattccaagaacacgctgtatctgcaaatgaacagcctgcgtgccgaggacaccgcggtatattactgtgcgaaatttccgtttactcatggtaagtttgactactggggtcagggaaccctggtcaccgtctcgagc

SEQ ID NO: 49 - protein, 289 aa, Artificial Sequence
  Amino acid sequence identified using molecular biology techniques.
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKETVAAPSEVQLLESGGGLVQPGGSLRLSCTASGFTFRHYRMGWVRQAPGKGLEWVSWIRPDGTFTYYADSVKGRFTISRDNSKNTLYLQMNSLRAEDAAVYYCAKSYMSGTFDYWGQGTLVTVSS

SEQ ID NO: 50 - DNA, 867 bp, Artificial Sequence
  Nucleic acid sequence identified using molecular biology techniques.
tgcgacttgccacagacacatagtttgggatcaagaagaacattgatgttattagcacaaatgcgtagaatttctttgttctcttgtctaaaggaccgtcacgacttcggattccctcaggaagagtttggaaaccaattccaaaaagcagaaactattcctgtcttgcacgaaatgatccagcaaatattcaatttgttttctacaaaggactcatcagccgcttgggatgaaactctgttagataaattctacactgaactatatcaacaactgaacgatctagaggcttgcgttattcagggtgtaggagttactgaaactcccctaatgaaagaagattcaattctagccgttagaaaatactttcagcgtatcacattgtatttaaaggaaaagaaatactccccatgtgcatgggaggtggttagagcagaaattatgaggtccttctctctttctacgaatttgcaagaatctttgagatctaaggaaaccgtcgctgctccatctgaggtgcagctgttggagtctgggggaggcttggtacagcctggggggtccctgcgtctctcctgtacagcctccggattcacctttaggcattatcgtatgggttgggtccgccaggctccagggaagggtctagagtgggtctcatggattcgtccggatggtacgtttacatactacgcagactccgtgaagggccggttcaccatctcccgcgacaattccaagaacacgctgtatctgcaaatgaacagcctgcgtgccgaggacgctgcggtatattactgtgcgaaatcttatatgtctggtacttttgactactggggtcagggaaccctggtcaccgtctcgagc

SEQ ID NO: 51 - protein, 288 aa, Artificial Sequence
  Amino acid sequence identified using molecular biology techniques.
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKETVAAPSEVQLLESGGGLVQPGGSLRLSCAASGFTFNHYTMGWVRQAPGKGREWVSLIHPSGTVTYYADSVKGRFTISRDNSKNTLYLQMNSLRAEDTAVYYCAKWERRTFDYWGQGTLVTVSS

SEQ ID NO: 52 - DNA, 864 bp, Artificial Sequence
  Nucleic acid sequence identified using molecular biology techniques.
tgcgacttgccacagacacatagtttgggatcaagaagaacattgatgttattagcacaaatgcgtagaatttctttgttctcttgtctaaaggaccgtcacgacttcggattccctcaggaagagtttggaaaccaattccaaaaagcagaaactattcctgtcttgcacgaaatgatccagcaaatattcaatttgttttctacaaaggactcatcagccgcttgggatgaaactctgttagataaattctacactgaactatatcaacaactgaacgatctagaggcttgcgttattcagggtgtaggagttactgaaactcccctaatgaaagaagattcaattctagccgttagaaaatactttcagcgtatcacattgtatttaaaggaaaagaaatactccccatgtgcatgggaggtggttagagcagaaattatgaggtccttctctctttctacgaatttgcaagaatctttgagatctaaggaaaccgtcgctgctccatctgaggtgcagctgttggagtctgggggaggcttggtacagcctggggggtccctgcgtctctcctgtgcagcctccggattcacctttaatcattatacgatggggtgggtccgccaggctccagggaagggtcgagagtgggtctcattgattcatccgagtggtacggtgacatactacgcagactccgtgaagggccggttcaccatctcccgcgacaattccaagaacacgctgtatctgcaaatgaacagcctgcgtgccgaggacaccgcggtatattactgtgcgaaatgggagaggcggacttttgactactggggtcagggaaccctggtcaccgtctcgagc

SEQ ID NO: 53 - protein, 290 aa, Artificial Sequence [sequence omitted]

SEQ ID NO: 54 - DNA, 870 bp, Artificial Sequence
  Nucleic acid sequence identified using molecular biology techniques.
tgcgacttgccacagacacatagtttgggatcaagaagaacattgatgttattagcacaaatgcgtagaatttctttgttctcttgtctaaaggaccgtcacgacttcggattccctcaggaagagtttggaaaccaattccaaaaagcagaaactattcctgtcttgcacgaaatgatccagcaaatattcaatttgttttctacaaaggactcatcagccgcttgggatgaaactctgttagataaattctacactgaactatatcaacaactgaacgatctagaggcttgcgttattcagggtgtaggagttactgaaactcccctaatgaaagaagattcaattctagccgttagaaaatactttcagcgtatcacattgtatttaaaggaaaagaaatactccccatgtgcatgggaggtggttagagcagaaattatgaggtccttctctctttctacgaatttgcaagaatctttgagatctaaggaaaccgtcgctgctccatctgaggtgcagctgttggagtctgggggaggcttggtacagcctggggggtccctgcgtctctcctgtgcagcctccggattcacctttgatacgagtagtatgttgtgggtccgccaggctccagggaagggtctagagtgggtctcagttattcatcagagtggtacgcctacatactacgcagactccgtgaagggccggttcaccatctcccgcgacaattccaagaacacgctgtatctgcaaatgaacagcctgcgtgccgaggacaccgcggtatattactgtgcgaaatttccgtcttctaggatgaagtttgactactggggtcagggaaccctggtcaccgtctcgagc

SEQ ID NO: 55 - protein, 290 aa, Artificial Sequence
  Amino acid sequence identified using molecular biology techniques.
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKETVAAPSEVQLLESGGGLVQPGGSLRLSCAASGFTFDTSSMLWVRQAPGKGLEWVSVIHQSGTPTYYADSVKGRFTISRDNSKNTLYLQMNSLRAEDTAVYYCAKFPSRKMKFDYRGQGTLVTVSS

SEQ ID NO: 56 - DNA, 870 bp, Artificial Sequence
  Nucleic acid sequence identified using molecular biology techniques.
tgcgacttgccacagacacatagtttgggatcaagaagaacattgatgttattagcacaaatgcgtagaatttctttgttctcttgtctaaaggaccgtcacgacttcggattccctcaggaagagtttggaaaccaattccaaaaagcagaaactattcctgtcttgcacgaaatgatccagcaaatattcaatttgttttctacaaaggactcatcagccgcttgggatgaaactctgttagataaattctacactgaactatatcaacaactgaacgatctagaggcttgcgttattcagggtgtaggagttactgaaactcccctaatgaaagaagattcaattctagccgttagaaaatactttcagcgtatcacattgtatttaaaggaaaagaaatactccccatgtgcatgggaggtggttagagcagaaattatgaggtccttctctctttctacgaatttgcaagaatctttgagatctaaggaaaccgtcgctgctccatctgaggtgcagctgttggagtctgggggaggcttggtacagcctggggggtccctgcgtctctcctgtgcagcctccggattcacctttgatacgagtagtatgttgtgggtccgccaggctccagggaagggtctagagtgggtctcagttattcatcagagtggtacgcctacatactacgcagactccgtgaagggccggttcaccatctcccgcgacaattccaagaacacgctgtatctgcaaatgaacagcctgcgtgccgaggacactgcggtatattactgtgcgaaatttccgtctaggaagatgaagtttgactaccggggtcagggaaccctggtcaccgtctcgagc

SEQ ID NO: 57 - protein, 165 aa, Artificial Sequence [sequence omitted]

SEQ ID NO: 58 - DNA, 495 bp, Artificial Sequence
  Interferon alpha 2b nucleotide sequence
tgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaa

SEQ ID NO: 59 - DNA, 25 bp, Artificial Sequence
  Nucleic acid sequence identified using molecular biology techniques.
gcccggatccaccggctgtgatctg

SEQ ID NO: 60 - DNA, 30 bp, Artificial Sequence
  Nucleic acid sequence identified using molecular biology techniques.
ggaggatggagactgggtcatctggatgtc

SEQ ID NO: 61 - DNA, 25 bp, Artificial Sequence
  Nucleic acid sequence identified using molecular biology techniques.
gcccggatccaccggctgtgatctg

SEQ ID NO: 62 - protein, 20 aa, Artificial Sequence
  Amino acid sequence identified using molecular biology techniques.
METDTLLLWVLLLWVPGSTG

SEQ ID NO: 63 - DNA, 61 bp, Artificial Sequence
  Nucleic acid sequence identified using molecular biology techniques.
atggagaccgacaccctgctgctgtgggtgctgctgctgtgggtgcccggatccaccgggc